SEQUENCE LISTING (41 sequences)

SEQ ID NO: 1 - protein, 328 aa, Artificial Sequence
  Derived from Env Amphotropic Murine Leukaemia Virus 4070A
  PEPTIDE (1)..(328)
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLC

SEQ ID NO: 2 - protein, 357 aa, Artificial Sequence
  Derived from Env Gibbon Ape Leukaemia Virus
  PEPTIDE (1)..(357)
MVLLPGSMLLTSNLHHLRHQMSPGSWKRLIILLSCVFGGGGTSLQNKNPHQPMTLTWQVLSQTGDVVWDTKAVQPPWTWWPTLKPDVCALAASLESWDIPGTDVSSSKRVRPPDSDYTAAYKQITWGAIGCSYPRARTRMASSTFYVCPRDGRTLSEARRCGGLESLYCKEWDCETTGTGYWLSKSSKDLITVKWDQNSEWTQKFQQCHQTGWCNPLKIDFTDKGKLSKDWITGKTWGLRFYVSGHPGVQFTIRLKITNMPAVAVGPDLVLVEQGPPRTSLALPPPLPPREAPPPSLPDSNSTALATSAQTPTVRKTIVTLNTPPPTTGDRLFDLVQGAFLTLNATNPGATESCWLC

SEQ ID NO: 3 - protein, 239 aa, Artificial Sequence
  Derived from Env Feline endogenous Leukaemia Virus RD 114
  PEPTIDE (1)..(239)
MKLPTGMVILCSLIIVRAGFDDPRKAIALVQKQHGKPCECSGGQVSEAPPNSIQQVTCPGKTAYLMTNQKWKCRVTPKISPSGGELQNCPCNTFQDSMHSSCYTEYRQCRRINKTYYTATLLKIRSGSLNEVQILQNPNQLLQSPCRGSINQPVCWSATAPIHISDGGGPLDTKRVWTVQKRLEQIHKAMTPELQYHPLALPKVRDDLSLDARTFDILNTTFRLLQMSNFSLAQDCWLC

SEQ ID NO: 4 - protein, 462 aa, Artificial Sequence
  Derived from Vesicular Stomatitis Virus G glycoprotein
  PEPTIDE (1)..(462)
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWK

SEQ ID NO: 5 - protein, 339 aa, Artificial Sequence
  Derived from Env ecotropic Moloney -Murine Leukaemia Virus
  PEPTIDE (1)..(339)
MARSTLSKPLKNKVNPRGPLIPLILLMLRGVSTASPGSSPHQVYNITWEVTNGDRETVWATSGNHPLWTWWPDLTPDLCMLAHHGPSYWGLEYQSPFSSPPGPPCCSGGSSPGCSRDCEEPLTSLTPRCNTAWNRLKLDQTTHKSNEGFYVCPGPHRPRESKSCGGPDSFYCAYWGCETTGRAYWKPSSSWDFITVNNNLTSDQAVQVCKDNKWCNPLVIRFTDAGRRVTSWTTGHYWGLRLYVSGQDPGLTFGIRLRYQNLGPRVPIGPNPVLADQQPLSKPKPVKSPSVTKPPSGTPLSPTQLPPAGTENRLLNLVDGAYQALNLTSPDKTQECWLC

SEQ ID NO: 6 - protein, 349 aa, Artificial Sequence
  Derived from Env ecotropic Friend Murine Leukaemia Virus
  PEPTIDE (1)..(349)
MACSTLPKSPKDKIDPRDLLIPLILFLSLKGARSAAPGSSPHQVYNITWEVTNGDRETVWAISGNHPLWTWWPVLTPDLCMLALSGPPHWGLEYQAPYSSPPGPPCCSGSSGSSAGCSRDCDEPLTSLTPRCNTAWNRLKLDQVTHKSSEGFYVCPGSHRPREAKSCGGPDSFYCASWGCETTGRVYWKPSSSWDYITVDNNLTTSQAVQVCKDNKWCNPLAIQFTNAGKQVTSWTTGHYWGLRLYVSGRDPGLTFGIRLRYQNLGPRVPIGPNPVLADQLSLPRPNPLPKPAKSPPASNSTPTLISPSPTPTQPPPAGTGDRLLNLVQGAYQALNLTNPDKTQECWLC

SEQ ID NO: 7 - protein, 340 aa, Artificial Sequence
  Derived from Env ecotropic AKV Murine Leukaemia Virus
  PEPTIDE (1)..(340)
MESTTLSKPFKNQVNPWGPLIVLLILGGVNPVTLGNSPHQVFNLTWEVTNGDRETVWAITGNHPLWTWWPDLTPDLCMLALHGPSYWGLEYRAPFSPPPGPPCCSGSSDSTPGCSRDCEEPLTSYTPRCNTAWNRLKLSKVTHAHNGGFYVCPGPHRPRWARSCGGPESFYCASWGCETTGRASWKPSSSWDYITVSNNLTSDQATPVCKGNEWCNSLTIRFTSFGKQATSWVTGHWWGLRLYVSGHDPGLIFGIRLKITDSGPRVPIGPNPVLSDRRPPSRPRPTRSPPPSNSTPTETPLTLPEPPPAGVENRLLNLVKGAYQALNLTSPDKTQECWLC

SEQ ID NO: 8 - protein, 289 aa, Artificial Sequence
  Derived from Env M813 Murine Leukaemia Virus
  PEPTIDE (1)..(289)
MADSSLSEPSKDKTHSRAPTIALGILLVLGRVAQGGSPHQPVTLTWQVLDEELYVKWETSGKHPEWTWWPDLTPDLSGFSCDYSQLNVPDFYVCPGHGKSYSRRVCGGAESAFCAKWGCETTGDAYWNPNRPDLIIVKKGQNRTACKGNKCQGKYCNPLKITFTDQGKNSREWKRGLRWGCWVHLLSQHFIFYIRLQVTRSPVLAIGPNPVVADQKPPSRPAPVIPPVPPQVNPTGATDNTTGTTPTTVLSTKQPQRPGTGDRLLDLVQGAYLALNFTNPEKTQECWLC

SEQ ID NO: 9 - protein, 319 aa, Artificial Sequence
  Derived from Env 10A1 Murine Leukaemia Virus
  PEPTIDE (1)..(319)
MEGPAFSKPLKDKINPWKSLMVMGVYLRVGMAESPHQVFNVTWRVTNLMTGRTANATSLLGTVQDAFPRLYFDLCDLVGEEWDPSDQEPYVGYGCKYPGGRKRTRTFDFYVCPGHTVKSGCGGPREGYCGEWGCETTGQAYWKPTSSWDLISLKRGNTPWDTGCSKMACGPCYDLSKVSNSFQGATRGGRCNPLVLEFTDAGKKANWDGPKSWGLRLYRTGTDPITMFSLTRQVLNIGPRIPIGPNPVITGQLPPSRPVQIRLPRPPQPPPTGAASIVPETAPPSQQPGTGDRLLNLVEGAYQALNLTNPDKTQECWLC

SEQ ID NO: 10 - protein, 316 aa, Artificial Sequence
  Derived from Env Xenotropic Murine Leukaemia Virus (NZB)
  PEPTIDE (1)..(316)
MLVMEGSAFSKPLKDKINPWGPLIVMGILVRAGASVQRDSPHQIFNVTWRVTNLMTGQTANATSLLGTMTDTFPKLYFDLCDLVGDYWDDPEPDIGDGCRTPGGRRRTRLYDFYVCPGHTVPIGCGGPGEGYCGKWGCETTGQAYWKPSSSWDLISLKRGNTPKDQGPCYDSSVSSGVQGATPGGRCNPLVLEFTDAGRKASWDAPKVWGLRLYRSTGADPVTRFSLTRQVLNVGPRVPIGPNPVITDQLPPSQPVQIMLPRPPHPPPSGTVSMVPGAPPPSQQPGTGDRLLNLVEGAYQALNLTSPDKTQECWLC

SEQ ID NO: 11 - protein, 313 aa, Artificial Sequence
  Derived from Env Xenotropic Murine Leukaemia Virus (Bxv1)
  PEPTIDE (1)..(313)
MEGPAFSKPLKDKINPWGPLIVIGILVRAGASVQRDSPHQVFNVTWRVTNLMTGQTANATSLLGTMTDTFPKLYFDLCDLVGDHWDDPEPDIGDGCRSPGGRKRSRLYDFYVCPGHTVPIGCGGPGEGYCGKWGCETTGQAYWKPSSSWDLISLKRGNTPKDQGPCYDSSVSSGVQGATPGGRCNPLVLEFTDAGKKASWDAPKVWGLRLYRSTGADPVTRFSLTRQVLNVGPRVPIGPNPVITEQLPPSQPVQIMLPRPPHPPPSGAASMVPGAPPPSQQPGTGDRLLNLVKGAYQALNLTSPDRTQECWLC

SEQ ID NO: 12 - protein, 314 aa, Artificial Sequence
  Derived from Env XMRV (new Human Murine Leukaemia Virus-like)
  PEPTIDE (1)..(314)
MESPAFSKPLKDKINPWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQGATPGGRCNPLVLEFTDAGKRASWDAPKTWGLRLYRSTGADPVTLFSLTRQVLNVGPRVPIGPNPVITEQLPPSQPVQIMLPRPPRPPPSGAASMVPGAPPPSQQPGTGDRLLNLVEGAYQALNLTSPDKTQECWLC

SEQ ID NO: 13 - protein, 310 aa, Artificial Sequence
  Derived from Env MCF polytropic Murine Leukaemia Virus (MX27)
  PEPTIDE (1)..(310)
MEGPAFSKPLKDKINPWGPLIVLGILIRAGVSVQHDSPHQVFNVTWRVTNLMTGQTANATSLLGTMTDAFPKLYFDLCDLIGDDWDETGLGCRTPGGRKRARTFDFYVCPGHTVPTGCGGPREGYCGKWGCETTGQAYWKPSSSWDLISLKRGNTPRNQGPCYDSSAVSSDIKGATPGGRCNPLVLEFTDAGKKASWDGPKVWGLRLYRSTGTDPVTRFSLTRQVLNIGPRVPIGPNPVITDQLPPSRPVQIMLPRPPQPPPPGAASIVPETAPPSQQPGTGDRLLNLVDGAYQALNLTSPDKTQECWLC

SEQ ID NO: 14 - protein, 301 aa, Artificial Sequence
  Derived from Env MCF polytropic Murine Leukaemia Virus (MX33)
  PEPTIDE (1)..(301)
MEGPAFSKPLKDKINPWAPLIVLGILIRAGVSVPHDSPHQVFNVTWRVTNLMTGQTANATSLLGTMTDAFPKLYFDLCDLIGDDWDETGLGCRTPGGRKRARTFDFYVCPGHTVPTGCGGPREGYCGKWGCETTGQAYWKPSSSWDLISLKRGNTPRNQGPCYDSSAVSSGIQGATPGGRCNPLVLEFTDAGKKASWDGPKVWGLRLYRSTGTDPVTRFSLTRQVLNIGPRIPIGPNPVITDQLPPSRPVQIMLPRPPQPSPTGAASIQPGTGDRLLNLVDGAYQALNLTSPDKTQECWLC

SEQ ID NO: 15 - protein, 310 aa, Artificial Sequence
  Derived from Env MCF1233 polytropic Murine Leukaemia Virus
  PEPTIDE (1)..(310)
MEGPAFSKPLKDKINPWGPLIILGILIRAGVSVQHDSPHQVFNVTWRVTNLMTGQTANATSLLGTMTDAFPKLYFDLCDLIGDDWDETGLGCRTPGGRKRARTFDFYVCPGHTVPTGCGGPREGYCGKWGCETTGQAYWKPSSSWDLISLKRGNTPQNQGPCYDSSAVSSDIKGATPGGRCNPLVLEFTDAGKKASWDGPKVWGLRLYRPTGTDPVTRFSLTRRVLNIGPRVPIGPNPVIADQLPPSRPVQIMLPRPPQPPPPGASSIVPETAPPSQQPGTGDRLLNLVDGAYQALNLTSPDKTQECWLC

SEQ ID NO: 16 - protein, 348 aa, Artificial Sequence
  Derived from Env Mus dunni endogenous virus
  PEPTIDE (1)..(348)
MKKPTKTTGLWKPLITLLSFACVAGAPSITLDLGNHNPHAPVQQSWEVLNEKGDVVWVATAVHPPWTWWPDLTPDICKLAAGSPNWDLPDHTDLNNPPSEQKCVPNGVGSTTGCSGQFYRANLRAAQFYVCPGQGQKGKLQQECRGASDYFCGKWTCETTGEAYWKPSADWDLITVKRGSGYDKPNQGERNPYKYLDSGCALKNYSPPGPCKGKYCNPLLIKFTEKGKQARLSWLKGNRWGWRVYIPIRDPGFIFTIRLTVRDLAVTSIGPNKVLTEQAPPVAPAPPRVPAVPAPPTSRPYTVGPSLETTLASPPLLDTENRLVSLVQGAFLVLNRTNPNMTQSCWLC

SEQ ID NO: 17 - protein, 315 aa, Artificial Sequence
  Derived from Env Feline Leukaemia Virus A
  PEPTIDE (1)..(315)
MESPTHPKPSKDKTLSWNLAFLVGILFTIDIGMANPSPHQIYNVTWVITNVQTNTQANATSMLGTLTDAYPTLHVDLCDLVGDTWEPIVLNPTNVKHGARYSSSKYGCKTTDRKKQQQTYPFYVCPGHAPSLGPKGTHCGGAQDGFCAAWGCETTGEAWWKPTSSWDYITVKRGSSQDNSCEGKCNPLVLQFTQKGRQASWDGPKMWGLRLYRTGYDPIALFTVSRQVSTITPPQAMGPNLVLPDQKPPSRQSQTGSKVATQRPQTNESAPRSVAPTTMGPKRIGTGDRLINLVQGTYLALNATDPNKTKDCWLC

SEQ ID NO: 18 - protein, 335 aa, Artificial Sequence
  Derived from Env Feline Leukaemia Virus B
  PEPTIDE (1)..(335)
MESPTHPKPSKDKTLSWNLVFLVGILFTIDIGMANPSPHQVYNVTWTITNLVTGTKANATSMLGTLTDAFPTMYFDLCDIIGNTWNPSDQEPFPGYGCDQPMRRWQQRNTPFYVCPGHANRKQCGGPQDGFCAVWGCETTGETYWRPTSSWDYITVKKGVTQGIYQCSGGGWCGPCYDKAVHSSTTGASEGGRCNPLILQFTQKGRQTSWDGPKSWGLRLYRSGYDPIALFSVSRQVMTITPPQAMGPNLVLPDQKPPSRQSQIESRVTPHHSQGNGGTPGITLVNASIAPLSTPVTPASPKRIGTGDRLINLVQGTYLALNATDPNRTKDCWLC

SEQ ID NO: 19 - protein, 312 aa, Artificial Sequence
  Derived from Env Feline Leukaemia Virus C
  PEPTIDE (1)..(312)
MESPTHPKPSKDKTFPWNLVFLVGILFQIDMGMANPSPHQVYNVTWVITNVQTNSRANATSMLGTLTDAYPTLYVDLCDLVGDTWEPIAPDPRSWARYSSSTHGCKTTDRKKQQQTYPFYVCPGHAPSMGPKGTYCGGAQDGFCAAWGCETTGEAWWKPTSSWDYITVKRGSNQDNSCKGKCNPLVLQFTQKGRQASWDRPKMWGLRLYRSGYDPIALFSVSRQVMTITPPQAMGPNLVLPDQKPPSRQSQTKSKVTTQRPQITSSTPRSVASATMGPKRIGTGDRLINLVQGTYLALNATDPNKTKDCWLC

SEQ ID NO: 20 - protein, 331 aa, Artificial Sequence
  Derived from Env Koala Retrovirus
  PEPTIDE (1)..(321)
MLLISNPRHLGHPMSPGNWKRLIILLSCVFGGAEMNQQHNNPHQPMTLTWQVLSQTGSVVWEKKAVEPPWTWWPSLEPDVCALVAGLESWDIPELTASASQQARPPDSNYEHAYNQITWGTLGCSYPRARTRIARSQFYVCPRDGRSLSEARRCGGLESLYCKEWGCETAGTAYWQPRSSWDLITVGQGHPTGTCERTGWCNPLKIEFTEPGKRFRNWLQGRTWGLRFYVTGHPGVQLTIRLVITSPPPVVVGPDPVLAEQGPPRKIPFLPRVPVPTLSPPASPIPTVQASPPAPSTPSPTTGDRLFGLVQGAFLALNATNPEATESCWLC

SEQ ID NO: 21 - protein, 329 aa, Artificial Sequence
  Derived from Env Porcine Endogeneous Retrovirus-A
  PEPTIDE (1)..(329)
MHPTLSRRHLPIRGGKPKRLKIPLSFASIAWFLTLSITPQVNGKRLVDSPNSHKPLSLTWLLTDSGTGININSTQGEAPLGTWWPELYVCLRSVIPGLNDQATPPDVLRAYGFYVCPGPPNNEEYCGNPQDFFCKQWSCITSNDGNWKWPVSQQDRVSYSFVNNPTSYNQFNYGHGRWKDWQQRVQKDVRNKQISCHSLDLDYLKISFTEKGKQENIQKWVNGISWGIVYYGGSGRKKGSVLTIRLRIETQMEPPVAIGPNKGLAEQGPPIQEQRPSPNPSDYNTTSGSVPTEPNITIKTGAKLFSLIQGAFQALNSTTPEATSSCWLC

SEQ ID NO: 22 - protein, 326 aa, Artificial Sequence
  Derived from Env Porcine Endogeneous Retrovirus-B
  PEPTIDE (1)..(326)
MHPTLSWRHLPTRGGEPKRLRIPLSFASIAWFLTLTITPQASSKRLIDSSNPHRPLSLTWLIIDPDTGVTVNSTRGVAPRGTWWPELHFCLRLINPAVKSTPPNLVRSYGFYCCPGTEKEKYCGGSGESFCRRWSCVTSNDGDWKWPISLQDRVKFSFVNSGPGKYKVMKLYKDKSCSPSDLDYLKISFTEKGKQENIQKWINGMSWGIVFYKYGGGAGSTLTIRLRIETGTEPPVAVGPDKVLAEQGPPALEPPHNLPVPQLTSLRPDITQPPSNGTTGLIPTNTPRNSPGVPVKTGQRLFSLIQGAFQAINSTDPDATSSCWLC

SEQ ID NO: 23 - protein, 305 aa, Artificial Sequence
  Derived from Env Human Endogeneous Retrovirus-T
  PEPTIDE (1)..(305)
MGPEAWVRPLKTAPKPGEAIRLILFIYLSCFFLPVMSSEPSYSFLLTSFTTGRVFANTTWRAGTSKEVSFAVDLCVLFPEPARTHEEQHNLPVIGAGSVDLAAGFGHSGSQTGCGSSKGAEKGLQNVDFYLCPGNHPDASCRDTYQFFCPDWTCVTLATYSGGSTRSSTLSISRVPHPKLCTRKNCNPLTITVHDPNAAQWYYGMSWGLRLYIPGFDVGTMFTIQKKILVSWSSPKPIGPLTDLGDPIFQKHPDKVDLTVPLPFLVPRPQLQQQHLQPSLMSILGGVHHLLNLTQPKLAQDCWLC

SEQ ID NO: 24 - protein, 189 aa, Artificial Sequence
  Derived from Env Human Endogeneous Retrovirus-W
MALPYHIFLFTVVSPSFTLTAPPPCRCMTSSSPYQEFLWRMQRPGNIDAPSYRSLCKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWIC

SEQ ID NO: 25 - skipped

SEQ ID NO: 26 - protein, 283 aa, Artificial Sequence
  Derived from Env Human Endogeneous Retrovirus-F
  PEPTIDE (1)..(283)
MNSPCDRLQQFIQVLLEESWSFPSFANTLHWPENLLSYIDELVWQGSLQNFHQHEVRFDKPPLRLPLTGFSSLTENWSSRQAVSSRLVATAASPPAGCQAPIAFLGLKFSSLGPARKNPALCFLYDQSNSKCNTSWVKENVGCPWHWCNIHEALIRTEKGSDPMFYVNTSTGGRDGFNGFNLQISDPWDPRWASGVDGGLYEHKTFMYPVAKIRIARTLKTTVTGLSDLASSIQSAEKELTSQLQPAADQAKSSRFSWLTLISEGAQLLQSTGVQNLSHCFLC

SEQ ID NO: 27 - protein, 228 aa, Artificial Sequence
  Derived from Env Human T Leukaemia Virus-1
  PEPTIDE (1)..(228)
MGKFLATLILFFQFCPLIFGDYSPSCCTLTIGVSSYHSKPCNPAQPVCSWTLDLLALSADQALQPPCPNLVSYSSYHATYSLYLFPHWTKKPNRNGGGYYSASYSDPCSLKCPYLGCQSWTCPYTGAVSSPYWKFQHDVNFTQEVSRLNINLHFSKCGFPFSLLVDAPGYDPIWFLNTEPSQLPPTAPPLLPHSNLDHILEPSIPWKSKLLTLVQLTLQSTNYTCIVC

SEQ ID NO: 28 - protein, 224 aa, Artificial Sequence
  Derived from Env Human T Leukaemia Virus-2
  PEPTIDE (1)..(224)
MGNVFFLLLFSLTHFPLAQQSRCTLTVGISSYHSSPCSPTQPVCTWNLDLNSLTTDQRLHPPCPNLITYSGFHKTYSLYLFPHWIKKPNRQGLGYYSPSYNDPCSLQCPYLGCQSWTCPYTGPVSSPSWKFHSDVNFTQEVSQVSLRLHFSKCGSSMTLLVDAPGYDPLWFITSEPTQPPPTSPPLVHDSDLEHVLTPSTSWTTKILKFIQLTLQSTNYSCMVC

SEQ ID NO: 29 - protein, 228 aa, Artificial Sequence
  Derived from Env Simian T Leukaemia Virus-3
  PEPTIDE (1)..(228)
MGKSGLYFSLICFYTLFPSSFGNPSRCTLFIGASSYHSDPCGSNHPRCTWRLDLFSLTKDQSLSPPCPGLVTYSQYHKPYSLYVFPHWIAKPDRRGLGYYSASYSDPCAIQCPYLGCQSWTCPYTGPVSSPHWKYTSDLNFTQEVSSISLHLHFSKCGYSFSFLLDAPGYDPVWLLSSQATQIPPTPAPLIRDPDLQHILEPSIPWSSKILNLILLALKSTNYSCMVC

SEQ ID NO: 30 - protein, 215 aa, Artificial Sequence
  Derived from Env Bovine Leukaemia Virus
  PEPTIDE (1)..(215)
MPKERRSRRRPQPIIRWVSLTLTLLALCQPIQTWRCSLSLGNQQWMTTYNQEAKFFISIDQILEAHNQSPFCPRSPRYTLDFVNGYPKIYWPPPQGRRRFGARAMVTYDCEPRCPYVGADHFDCPHWDNASQADQGSFYVNHQTLFLHLKQCHGIFTLTWEIWGYDPLITFSLHKIPDPPQPDFPQLNSDWVPSVRSWALLLNQTARAFPDCAIC

SEQ ID NO: 31 - protein, 224 aa, Artificial Sequence
  Derived from Env Human T Leukaemia Virus-4
  PEPTIDE (1)..(224)
MGNVLFLTLLATLGIPVLQASRCTITVGISSYHSSPCSPAQPLCTWALDLVSITKDQLLYPPCQNLITYSNYHKTYSLYLFPHWVQKPLRRGLGYYSASYSDPCSLQCPYLGSQSWTCPYTGPVSSPTWRFSTDVNFTQEVSRVSLKLHFSKCGSSLTLLIDAPGYDPLWYLTSEPTQEPPTPPPLVSDSDLEHVLTPSASWASKMLTLIHLTLQSTNYSCMVC

SEQ ID NO: 32 - protein, 244 aa, Artificial Sequence
  Derived from Env Amphotropic Murine Leukaemia Virus 4070A
  PEPTIDE (1)..(244)
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIG

SEQ ID NO: 33 - protein, 222 aa, Artificial Sequence
  Derived from Env Feline endogenous Leukaemia Virus RD 114
  PEPTIDE (1)..(222)
MKLPTGMVILCSLIIVRAGFDDPRKAIALVQKQHGKPCECSGGQVSEAPPNSIQQVTCPGKTAYLMTNQKWKCRVTPKISPSGGELQNCPCNTFQDSMHSSCYTEYRQCRRINKTYYTATLLKIRSGSLNEVQILQNPNQLLQSPCRGSINQPVCWSATAPIHISDGGGPLDTKRVWTVQKRLEQIHKAMTPELQYHPLALPKVRDDLSLDARTFDILNTTF

SEQ ID NO: 34 - protein, 296 aa, Artificial Sequence
  Derived from Env Xenotropic Murine Leukaemia Virus (NZB)
  PEPTIDE (1)..(296)
MLVMEGSAFSKPLKDKINPWGPLIVMGILVRAGASVQRDSPHQIFNVTWRVTNLMTGQTANATSLLGTMTDTFPKLYFDLCDLVGDYWDDPEPDIGDGCRTPGGRRRTRLYDFYVCPGHTVPIGCGGPGEGYCGKWGCETTGQAYWKPSSSWDLISLKRGNTPKDQGPCYDSSVSSGVQGATPGGRCNPLVLEFTDAGRKASWDAPKVWGLRLYRSTGADPVTRFSLTRQVLNVGPRVPIGPNPVITDQLPPSQPVQIMLPRPPHPPPSGTVSMVPGAPPPSQQPGTGDRLLNLVQ

SEQ ID NO: 35 - protein, 232 aa, Artificial Sequence
  Derived from Env Feline Leukaemia Virus C
  PEPTIDE (1)..(232)
MESPTHPKPSKDKTFPWNLVFLVGILFQIDMGMANPSPHQVYNVTWVITNVQTNSRANATSMLGTLTDAYPTLYVDLCDLVGDTWEPIAPDPRSWARYSSSTHGCKTTDRKKQQQTYPFYVCPGHAPSMGPKGTYCGGAQDGFCAAWGCETTGEAWWKPTSSWDYITVKRGSNQDNSCKGKCNPLVLQFTQKGRQASWDRPKMWGLRLYRSGYDPIALFSVSRQVMTITPPQ

SEQ ID NO: 36 - protein, 253 aa, Artificial Sequence
  Derived from Env Koala Retrovirus
  PEPTIDE (1)..(253)
MLLISNPRHLGHPMSPGNWKRLIILLSCVFGGAEMNQQHNNPHQPMTLTWQVLSQTGSVVWEKKAVEPPWTWWPSLEPDVCALVAGLESWDIPELTASASQQARPPDSNYEHAYNQITWGTLGCSYPRARTRIARSQFYVCPRDGRSLSEARRCGGLESLYCKEWGCETAGTAYWQPRSSWDLITVGQGHPTGTCERTGWCNPLKIEFTEPGKRFRNWLQGRTWGLRFYVTGHPGVQLTIRLVITSPPPVVVG

SEQ ID NO: 37 - protein, 309 aa, Artificial Sequence
  Derived from Env Porcine Endogeneous Retrovirus-A
  PEPTIDE (1)..(309)
MHPTLSRRHLPIRGGKPKRLKIPLSFASIAWFLTLSITPQVNGKRLVDSPNSHKPLSLTWLLTDSGTGININSTQGEAPLGTWWPELYVCLRSVIPGLNDQATPPDVLRAYGFYVCPGPPNNEEYCGNPQDFFCKQWSCITSNDGNWKWPVSQQDRVSYSFVNNPTSYNQFNYGHGRWKDWQQRVQKDVRNKQISCHSLDLDYLKISFTEKGKQENIQKWVNGISWGIVYYGGSGRKKGSVLTIRLRIETQMEPPVAIGPNKGLAEQGPPIQEQRPSPNPSDYNTTSGSVPTEPNITIKTGAKLFSLIQ

SEQ ID NO: 38 - protein, 239 aa, Artificial Sequence
  Derived from Env Porcine Endogeneous Retrovirus-B
MHPTLSWRHLPTRGGEPKRLRIPLSFASIAWFLTLTITPQASSKRLIDSSNPHRPLSLTWLIIDPDTGVTVNSTRGVAPRGTWWPELHFCLRLINPAVKSTPPNLVRSYGFYCCPGTEKEKYCGGSGESFCRRWSCVTSNDGDWKWPISLQDRVKFSFVNSGPGKYKVMKLYKDKSCSPSDLDYLKISFTEKGKQENIQKWINGMSWGIVFYKYGGGAGSTLTIRLRIETGTEPPVAVG

SEQ ID NO: 39 - skipped

SEQ ID NO: 40 - protein, 178 aa, Artificial Sequence
  Derived from Env Human T Leukaemia Virus-2
  PEPTIDE (1)..(178)
MGNVFFLLLFSLTHFPLAQQSRCTLTVGISSYHSSPCSPTQPVCTWNLDLNSLTTDQRLHPPCPNLITYSGFHKTYSLYLFPHWIKKPNRQGLGYYSPSYNDPCSLQCPYLGCQSWTCPYTGPVSSPSWKFHSDVNFTQEVSQVSLRLHFSKCGSSMTLLVDAPGYDPLWFITSEPTQ

SEQ ID NO: 41 - protein, 181 aa, Artificial Sequence
  Derived from Env Bovine Leukaemia Virus
  PEPTIDE (1)..(181)
MPKERRSRRRPQPIIRWVSLTLTLLALCQPIQTWRCSLSLGNQQWMTTYNQEAKFFISIDQILEAHNQSPFCPRSPRYTLDFVNGYPKIYWPPPQGRRRFGARAMVTYDCEPRCPYVGADHFDCPHWDNASQADQGSFYVNHQTLFLHLKQCHGIFTLTWEIWGYDPLITFSLHKIPDPPQ

(19) United States
(12) Patent Application Publication
(10) Pub. No.: US 2015/0099653 A1
(43) Pub. Date: Apr. 9, 2015

(54) PROCESS OF DIAGNOSTIC, PROGNOSTIC AND THERAPEUTIC MONITORING OF SOLID TUMORS

(71) Applicants: CENTRE NATIONAL DE LA RECHERCHE SCIENTIFIQUE, PARIS (FR); UNIVERSITE MONTPELLIER 2 SCIENCES ET TECHNIQUES, MONTPELLIER CEDEX 5 (FR); INSTITUT CURIE, PARIS CEDEX 05 (FR)

(72) Inventors: Jean-Luc Battini, Montpellier (FR); Didier Decaudin, Verrieres Le Buison (FR); Gerald Massonnet, Conde-Sur Vesgre (FR); Vincent Petit, Paris (FR); Marc Sitbon, Montpellier (FR)

(21) Appl. No.: 14/354,246

(22) PCT Filed: Oct. 29, 2012
(86) PCT No.: PCT/EP2012/071408

§ 371 Date: Apr. 25, 2014

Related U.S. Application Data
(60) Provisional application No. 61/552,594, filed on Oct. 28, 2011.

(51) Int. Cl. G01N 33/574; C12N 5/071

(52) U.S. Cl. CPC G01N 33/57415; C12N 5/0602; G01N 2333/705; G01N 2500/10
USPC 506/9; 435/381; 435/7.23; 506/18

(57) ABSTRACT

A process of diagnostic, prognostic and therapeutic monitoring of solid tumors, and new biological markers of tumor.

The present invention relates to a process of diagnostic, prognostic and therapeutic monitoring of solid tumors.

The invention also relates to new biological markers of tumor.

Tumor cell metabolism is of great importance in both basic and clinical cancer research. Understanding the molecular mechanisms of cancer cell metabolism could help guide drug discovery and development, as well as clinical evaluation and treatment of patient disease (Hsu P P, Sabatini D M. Cancer cell metabolism: Warburg and beyond. Cell. 2008 Sep. 5; 134(5):703-7; Hanahan D, Weinberg R A. Hallmarks of cancer: the next generation. Cell. 2011 Mar. 4; 144(5):646-74). Metabolomic approaches used to date rely on quantification of end products (Rubakhin S S, Romanova E V, Nemes P, Sweedler J V. Profiling metabolites and peptides in single cells. Nat Methods. 2011 April; 8(4 Suppl):520-9) of anabolism and catabolism. Measurement of the metabolite transporters themselves, however, would be of interest as they are key players in cell metabolism, controlling the influx of nutrients and efflux of toxic metabolic side products (Ganapathy V, Thangaraju M, Prasad P D. Nutrient transporters in cancer: relevance to Warburg hypothesis and beyond. Pharmacol Ther. 2009 January; 121(1):29-40). Cancer cells are highly dependent on metabolic activity, and the monitoring of these metabolite transporters expression profiles would be of prime interest.

Gamma and delta retroviruses have evolved to adapt membrane metabolite transporters as receptors for viral entry into the cell. Entry is mediated by the Receptor Binding Domain (RBD) of the viral envelope subunit (Env SU) which binds to the metabolite transporter receptor. Retroviral envelope-derived probes were designed as specific ligands to bind and quantitate the extracellular domains of defined sets of metabolite transporters (Manel N, Kim F J, Kinet S, Taylor N, Sitbon M, Battini J L. The ubiquitous glucose transporter GLUT-1 is a receptor for HTLV. Cell 2003 Nov. 14; (1154):449-59; Manel N, Kinet S, Battini J L, Kim F J, Taylor N, Sitbon M. The HTLV receptor is an early T-cell activation marker whose expression requires de novo protein synthesis. Blood 2003 Mar. 1; 101(5):1913-8; Montel-Hagen A, Kinet S, Manel N, Mongellaz C, Prohaska R, Battini J L, Delaunay J, Sitbon M, Taylor N. Erythrocyte Glut1 triggers dehydroascorbic acid uptake in mammals unable to synthesize vitamin C. Cell 2008 Mar. 21; 132(6):1039-48; Montel-Hagen A, Blanc L, Boyer-Clavel M, Jacquet C, Vidal M, Sitbon M, Taylor N. The Glut1 and Glut4 glucose transporters are differentially expressed during perinatal and postnatal erythropoiesis. Blood 2008 Dec. 1; 112(12):4729-38; Swainson L, Kinet S, Manel N, Battini J L, Sitbon M, Taylor N. Glucose transporter 1 expression identifies a population of cycling CD4+ CD8+ human thymocytes with high CXCR4-induced chemotaxis. Proc Natl Acad Sci USA 2005 Sep. 6; 102(36):12867-72, Lagrue E, Abe H, Lavanya M, Touhami J, Bodard S, Chalon S, Battini J L, Sitbon M, Castelnau P. Regional characterization of energy metabolism in the brain of normal and MPTP-intoxicated mice using new markers of glucose and phosphate transport. J Biomed Sci. 2010 Dec. 4; 17:91). Surface labelling with these ligands is easily performed on cultured cell lines or circulating hematopoietic cells. It is much more difficult, however, to obtain and label single cells from solid tumors. This requires development and validation of methods that will produce viable cells with intact antigens and membrane metabolite transporters, representative of the original tumor composition. Retroviral envelope-derived probes, which can be used for specific, high-affinity tagging of metabolic transporters on human cells, have been disclosed in WO 2010/079208. These transporters carry a wide variety of metabolites, including, but not limited to: neutral amino acids (AA), cationic AA, glucose, inorganic phosphate, potassium ions, heme and vitamins.

Retroviral envelope-derived probes of WO 2010/079208 have been used for the detection of membrane receptors present in a target cell such as haematopoietic stem cells, such as CD34 cells, or differentiated cells such as B-cells or T-cells.

Technical challenges encountered in multiparametric analysis of single viable solid tissue cells are often the same, regardless of the origin of the tissue: (i) total cell yield recovery must be maximised to produce sufficient and representative material for experiments, dramatically important for identification of small populations; (ii) cell viability and integrity are major concerns when cells are used for in vitro culture, injection into animals or molecular analysis. Apoptosis in the dissociated cells should be carefully assessed; (iii) cell membrane antigens must remain intact. Poor or selective tissue dissociation can bias the composition of cell subpopulations analysed and, consequently, the results.

Single viable cell recovery protocols include two main steps: tissue dissociation and cell purification. Tissue dissociation involves mechanical dissociation followed by enzyme digestion, both of which are detrimental to cells. Mechanical disaggregation with a scalpel to mince tissue into small pieces is necessary to increase the tissue surface accessibility to enzymes. Enzyme cocktails must be carefully chosen and tailored to the tissue, particularly for epithelial carcinomas in which the epithelial junctions (zonula occludens) are much more difficult to disrupt than the contiguous mesenchymal or stromal tissue. Looking at the relative viability, trypsin is the most potent enzyme for cell dissociation, but short incubation time results in poor total recovery yield, and some antigens of interest regarding cell phenotyping and/or sorting are sensitive to tryptic activity (Limited loss of nine tumor-associated surface antigenic determinants after tryptic cell dissociation Corver, W E Cytometry. 1995 Mar. 1; 19(3):267-72). Similarly, other enzymes such as collagenases, dispase, or hyaluronidase are often used in customised cocktails. Once single cells have been obtained, purification of interest cells has to be performed from the vast majority of the material obtained at this step, consisting in red cells, necrotic components, and debris. Red cell lysis is often realised using NH₄Cl based buffer, with a possible toxicity for nucleated cells (Responses in primary astrocytes and C6-glioma cells to ammonium chloride and dibutyryl cyclic-AMP Haghighat, N Neurochem Res. 2000 February; 25(2):277-84). Red cells, dead cells, and debris could also be eliminated using a Ficoll™ gradient (Davidson, W F A procedure for removing red cells and dead cells from lymphoid cell suspensions J Immunol Methods. 1975 June; 7(2-3):291-300; Rubenstein, M Isolation of viable rat ventral prostate epithelial and nonepithelial cells Endocrinology. 1980 February; 106(2):530-40), but the final cell recovery will depend on the density gradient. Indeed, tumor cells are constituted by a mix of aneuploid and polyploid cells, with the latter ones lost through commonly used single Ficoll™ density.

One of the aims of the invention is to provide an improved recovery process of single viable cells from a solid tumor giving both a higher yield of the number of viable cells per gram of tumor and an increased percentage of viability, suitable for cell surface component analyses.

Another aim of the invention is to use at least one receptor binding ligand comprising the RBD for the identification and detection of membrane receptors present on the surface of single viable cells obtained or not with the improved recovery process of single viable cells from a solid tumor of the invention.

Another aim of the invention is to provide a process of diagnostic and prognostic of a solid tumor in a patient.

Another aim of the invention is to provide a process of therapeutic response assessment in a patient having a treatment against a solid tumor by means of RBD and a recovery process of single viable cells from a solid tumor.

Another aim of the invention is to provide a process of therapeutic response assessment in a patient having a treatment against a solid tumor by means of RBD and the improved recovery process of single viable cells from a solid tumor of the invention.

Still another aim of the invention is to provide a screening process of molecules active against a solid tumor by means of RBD and the improved recovery process of single viable cells from a solid tumor of the invention.

The present invention relates to a recovery process of single viable cells from a solid tumor comprising two tissue dissociation steps using a non enzymatic dissociation buffer (NEDB) and an enzymatic tissue dissociation, in particular consisting of collagenase III and DNase I to obtain a mixture of isolated dead or viable cells and debris, followed by a cell purification step with a dual density Ficoll™ to eliminate red cells and debris, and thus enrich said mixture in single viable cells.

The expression “single viable cells” means that said cells are substantially enriched in nucleated cells (constituted of both polyploid and aneuploid cells), by eliminating specifically a major part of debris, necrotic components, dead cells and red cells.

Live cells could be further identified and analyzed by flow cytometry.

The expression “solid tumor” refers to a vertebrate solid tumor, i. e. a mass of cells that grows over time and can be a benign, pre-malignant or malignant tumor.

Solid tumors are localized in a particular organ, tissue or gland—for example, in the breast, the pancreas, the uterus, the cervix, the vagina, the vulva, the ovary, the trophoblast, the prostate, the testis, the penis, the ureter, the bladder, the urethra, the mouth, the throat, the oesophagus, the stomach, the colon, the rectum, the instestine, the lungs, the thymus, the kidney, the adrenal gland, the muscles, the thyroid, the parathyroid, the skin, the liver, the bone, the brain, the eye, . . . .

Non enzymatic dissociation buffer (NEDB) is a chelator cocktail used for the tissue dissociation.

The purification step with a dual Ficoll™ allows to enrich the mixture obtained after the two dissociation steps in nucleated cells constituted of aneuploid and polyploid cells in contrast to prior art wherein polyploid cells, that are a characteristic feature of many cancer tumor, can be lost with the commonly used Ficoll™ density gradients.

The expression “enrich single viable cells” means therefore that said single viable cells are further enriched in nucleated cells.

The inventors have thus found that combining two dissociation steps, one enzymatic and the other one non enzymatic, enzymatic dissociation being carried out before or after the non enzymatic dissociation, followed by the cell purification with a dual density Ficoll™, gives both a higher yield of the number of viable cells per gram of tumor and an increased percentage of viability.

Another advantage of the invention is to keep polyploid cells in the mixture constituting the single viable cells.

In an advantageous embodiment, the number of viable cells per gram of tumor, after the two tissue dissociation steps is comprised from 1 to 100×10⁶ cells, preferably from 10 to 100×10⁶ cells depending on the tumor.

The number of viable cells is given per gram of the tumor before said dissociation steps.

In an advantageous embodiment, the percentage of viable cells per gram of tumor after the two tissue dissociation steps is comprised from 5 to 90%, preferably from 10 to 60%, depending on the tumor.

The percentage of viable cells is given by the ratio between live cells and total cells (live and dead cells, as assessed by trypan blue exclusion or any other viability dye).

In an advantageous embodiment, the number of viable cells per gram of tumor after the cell purification step is comprised from 0.5 to 100×10⁶ cells, preferably from 1 to 100×10⁶ cells depending on the tumor.

The number of viable cells is given per gram of the tumor before said dissociation steps.

In an advantageous embodiment, the percentage of viable cells per gram of tumor after the two tissue dissociation steps is comprised from 5 to 95%, preferably from 30 to 90%, depending on the tumor.

The percentage of viable cells is given by the ratio between live cells and total cells (live and dead cells, as assessed by trypan blue exclusion or any other viability dye).

In an advantageous embodiment, the present invention relates to a recovery process of single viable cells from a solid tumor defined above, comprising further an enzymatic tissue dissociation step with trypsin after said two tissue dissociation steps to obtain a mixture of isolated dead or viable cells and debris.

The treatment with trypsin allows improving the percentage of viability after the dissociation steps.

In an advantageous embodiment, the present invention relates to a recovery process of single viable cells from a solid tumor, comprising further or not an enzymatic tissue dissociation step with trypsin, as defined above, wherein said solid tumor is a human solid tumor.

In this embodiment, single viable cells obtained are therefore only of human origin.

In an advantageous embodiment, the present invention relates to a recovery process of single viable cells from a solid tumor, comprising further or not an enzymatic tissue dissociation step with trypsin, as defined above, wherein said solid tumor is a human solid tumor previously grafted in a mouse.

In this embodiment, a human solid tumor excised from a patient has been previously grafted in a mouse by techniques well known from a man skilled in the art. Said mouse is called a xenografted mouse and is used as a preclinical model.

Nevertheless, single viable cells obtained are only of human origin.

In an advantageous embodiment, the present invention relates to a recovery process of single viable cells from a solid tumor, comprising further or not an enzymatic tissue dissociation step with trypsin, as defined above, wherein said human solid tumor or said grafted human solid tumor in a mouse, is a human breast cancer tumor or an UvMel melanoma.

In this embodiment, the solid tumor is malignant and is a human breast cancer or an UvMel melanoma, excised or previously excised from a patient, or a mouse in which the solid tumor has been previously grafted.

Several types of breast cancer tumor exist in human and thus, this embodiment covers all the human types known.

UvMel melanoma is a uveal melanoma such as a choroidal melanoma.

In an advantageous embodiment, the present invention relates to a recovery process of single viable cells from a solid tumor, comprising further or not an enzymatic tissue dissociation step with trypsin, as defined above, wherein said human solid tumor or said grafted human solid tumor, is a human breast cancer tumor selected from the group consisting of: HBCx-3, HBCx-4A, HBCx-8, HBCx-9, HBCx-10, HBCx-12A, HBCx-14, HBCx-22, HBCx-24, HBCx-30, HBCx-41, or an UVmel melanoma selected from the group consisting of MP34, MP38, MP41, MP42, MP46, MP47, MP55, MP71, MP77, MP80, MM26, MM33, MM52, MP65, MM66 and MP74, in particular MP33, MP34, MP41, MP55.

HBCx means human breast cancer and corresponds to xenografts of breast tumor from a patient.

MP means primar melanoma and MM means melanoma metastasis. They correspond to xenografts of UvMel melanomas from a patient

Characteristics of UvMel melanomas are given in FIG. 9.

In another aspect, the present invention relates to the use of single viable cells obtained from a recovery process of single viable cells from a solid tumor, in particular from a recovery process of single viable cells from a solid tumor defined above, in combination with at least one soluble receptor binding ligands derived from the soluble part of the glycoprotein of an enveloped virus that interacts with a cellular cognate receptor,
said receptor binding ligands containing a part or the totality of one of the receptor binding domains (RBD) of said glycoprotein, and,
said soluble receptor binding ligands being liable to interact with at least one membrane receptor of said single viable cells,
for the identification and quantification of the expression of membrane receptors present on the surface of said single viable cells, said identification and quantification taking place at a given time or during a given time interval, and allowing the clinical evaluation of patient's solid tumors relative to the diagnostic, prognostic or therapeutic response assessment.

In this aspect, single viable cells are obtained from a recovery process of single viable cells of the invention or are obtained by anyone of recovery processes existing in the literature comprising only one tissue dissociation, enzymatic or not enzymatic, and a purification step with a single high density Ficoll™, a single low density or dual density Ficoll™.

By the term “ligand” is meant a polypeptide.

The expression “derived from the soluble part of the glycoprotein of an enveloped virus” means that the receptor binding ligand is a fragment or a part of a glycoprotein contained in the envelope of a virus and can be obtained for example by cloning.

By the term “glycoprototein” is meant an envelope glycoprotein, a coat glycoprotein or a fusion glycoprotein.

The expression “that interacts with a cellular cognate receptor” means that the glycoprotein is liable to be recognized by a receptor present to the surface of a single viable cell.

One or more amino acids can be added to, deleted, or substituted from the peptidic sequence of this fragment or part of glycoprotein.

Receptor binding ligand containing part or the totality of the RBD can be chemically modified to add a fluorochrome.

RBD are found, in particular, in glycoprotein of the envelope of viruses, therefore, the receptor binding ligand contains the total RBD or a fragment or a part of said RBD.

Said part or totality of the RBD is liable to interact with at least one membrane receptor of a single viable cell.

RBD of the glycoprotein of the virus is able to bind to one or more membrane receptor(s) of a single viable cell.

By “membrane receptor” it is defined in the invention any protein or polypeptide anchored in the plasma membrane of cells. Said membrane receptor allows the interaction with glycoprotein of viruses.

Preferably the membrane receptors according to the invention are members of the multimembrane-spanning protein family which functions as transporters, such as nutriment and metabolite transporters, i.e. multimembrane-spanning proteins that allow the transport of nutriments, metals and metabolites across the plasma membrane.

The expression “said receptor binding ligand being liable to interact with at least one membrane receptor” means that said receptor binding ligand forms a complex with a receptor of the single viable cells by means of the RBD.

The soluble receptor binding ligand can also contain more than one RBD with its complete or partial sequence.

To obtain an interaction between the receptor and the membrane receptor of the single viable cells as defined above, the receptor binding ligand must be in a sufficient concentration to form a complex with the membrane receptor.

The expression “identification and the quantification of the expression of membrane receptors present on the surface of target cells” means that when a single viable cell expresses a membrane receptor, i.e. said receptor is present on the surface of the single viable cell, therefore a complex is formed between the membrane receptor of a biological interest target cell and the receptor binding ligand.

That complex can be detected if the receptor binding ligand has been for instance, but without being limited to, covalently coupled with a detectable molecule such as an antibody constant fragment (Fc) or a fluorescent compound (cyanins, alexa, quantum dots . . . )

That complex can also be detected if the receptor binding ligand has been tagged with different means well known by a person skilled in the art.

For instance, but without limitations, the tag used in the invention can be Hemaglutinin Tag, Poly Arginine Tag, Poly Histidine Tag, Myc Tag, Strep Tag, Flag Tag, S-Tag, HAT Tag, 3× Flag Tag, Calmodulin-binding peptide Tag, SBP Tag, Chitin-binding domain Tag, GST Tag, Maltose-Binding protein Tag, GFP and EGFP Tag, RFPs Tag, YFP Tag, CFP Tag, T7 tag, V5 tag, Xpress tag and all fluorescent molecules having an emission maximum comprised from 445 nm to 655 nm available from Olympus America Inc.

The use of a receptor binding ligand allows therefore on the one hand the identification of the receptor expressed on the target cell depending to the receptor binding ligand used and on the other hand the quantification of the complex formed, and thus the presence or not of a membrane receptor on the single viable cell and its quantification.

The expression “at a given time or during a given time interval” means that the detection and/or the quantification of the complex formed can be made just after the contacting of the receptor binding ligand and the membrane receptor of the target cell or after several minutes, in particular from 1 to 59 minutes, or several hours, in particular from 1 to 47 h, preferably 24 h, or days, in particular from 2 to 7 days, preferably 3 days, or several weeks, preferably 3 to 6 weeks when evaluating decay of said membrane receptors on the single viable cell, after said contacting, depending on the cells and the contacting conditions, in order to evaluate the modification of the expression of membrane receptors.

Contacting conditions include also the temperature that can vary from 0° C. to 37° C., in particular 0, 1, 2, 3 or 4° C., preferably near room temperature, in particular from 18° C. to 25° C., in particular 18, 19, 20, 21, 22, 23, 24 or 25° C., more preferably from 26 to 37° C., in particular 25, 26, 27, 28, 29, 30, 31, 32, 33, 34, 35, 36, or 37° C., preferably 30 or 37° C. depending on the target cells.

The inventors have found that in solid tumor, in particular solid cancer tumor, specific receptors are overexpressed or underexpressed at the surface of single viable cells from said solid tumor.

Thus the identification and the quantification of said receptors at the surface of single viable cells from said solid tumor allows to diagnostic and/or prognostic the presence of a malignant or not solid tumor.

The quantification of said receptors at the surface of single viable cells after several days or weeks or months of treatment of a patient having a malignant solid tumor allows to make an assessment of the therapeutic response of the patient and to evaluate the efficacy or not of said treatment.

Also of interest is the ability to distinguish between the different types of cells within the tumor: heterogeneous tumor cell clones, stem cells, inflammatory cells, stroma and peri-tumoral micro-environment.

In an advantageous embodiment, the present invention relates to the use of single viable cells obtained from a recovery process of single viable cells from a solid tumor, in particular from a recovery process of single viable cells from a solid tumor defined above, in combination with at least one soluble receptor binding ligands derived from the soluble part of the glycoprotein of an enveloped virus that interacts with a cellular cognate receptor,
said receptor binding ligands containing a part or the totality of one of the receptor binding domains (RBD) of said glycoprotein, and,
said soluble receptor binding ligands being liable to interact with at least one membrane receptor of said single viable cells,
for the identification and quantification of the expression of membrane receptors present on the surface of said single viable cells, said identification and quantification taking place at a given time or during a given time interval, and allowing the clinical evaluation of patient's solid tumors relative to the diagnostic, prognostic or therapeutic response assessment,
with the proviso that when only one soluble receptor binding ligands derived from PTLV is used, said membrane receptor which interacts with said receptor binding ligand is not Glut1.

In a advantageous embodiment, the present invention relates to the use of single viable cells obtained from a recovery process of single viable cells from a solid tumor, in particular from a recovery process of single viable cells from a solid tumor defined above, in combination with at least one soluble receptor binding ligands derived from the soluble part of the glycoprotein of an enveloped virus that interacts with a cellular cognate receptor,
said receptor binding ligands containing a part or the totality of one of the receptor binding domains (RBD) of said glycoprotein, and,
said soluble receptor binding ligands being liable to interact with at least one membrane receptor of said single viable cells,
for the identification and quantification of the expression of membrane receptors present on the surface of said single viable cells, said identification and quantification taking place at a given time or during a given time interval, and allowing the clinical evaluation of patient's solid tumors relative to the diagnostic, prognostic or therapeutic response assessment,
with the proviso that when only two soluble receptor binding ligands derived from PTLV are used, said membrane receptor which interacts with said receptor binding ligand is not Glut1 alone.

In an advantageous embodiment, the present invention relates to the use of single viable cells obtained from a recovery process of single viable cells from a solid tumor, in particular from a recovery process of single viable cells from a solid tumor defined above, wherein said receptor binding ligand is selected from the list consisting of: SEQ ID NO: 1 to 41.

The SEQ IDs 1 to 31 are constituted of the signal peptide when known, the receptor binding domain, the proline rich region (PRR) when known and the CXXC motif located downstream of the PRR.

The SEQ IDs 32 to 41 are constituted of the signal peptide when known, the receptor binding domain, and a part of the proline rich region (PRR).

In an advantageous embodiment, the present invention relates to the use of single viable cells obtained from a recovery process of single viable cells from a solid tumor, in particular from a recovery process of single viable cells from a solid tumor defined above, wherein said receptor binding ligand is selected from the list consisting of: SEQ ID NO: 1 to 41, and wherein said at least one soluble receptor binding ligand is a set of two soluble receptor binding ligands, and allows the identification and the quantification of the expression of at least two membrane receptors present on the surface of single viable cells.

In an advantageous embodiment, the present invention relates to the use of single viable cells obtained from a recovery process of single viable cells from a solid tumor, in particular from a recovery process of single viable cells from a solid tumor defined above, wherein said receptor binding ligand is selected from the list consisting of: SEQ ID NO: 1 to 41, and wherein said at least one soluble receptor binding ligand is a set of three to twelve soluble receptor binding ligands, in particular in particular three, four, five, six seven, eight, nine, ten, eleven, or twelve receptor binding ligands.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand derived from the soluble part of the glycoprotein of an enveloped virus is selected from the list consisting of: Amphotropic Murine Leukemia Retrovirus (AMLV, SEQ ID NO:32), Feline endogenous virus (RD114, SEQ ID NO:33), Xenotropic Murine Leukemia Virus (NZB, Xeno, SEQ ID NO: 34), Feline Leukemia Virus C (FeLVC, SEQ ID NO: 35), Koala Retrovirus (KoRV, SEQ ID NO: 36), Porcine Endogeneous Retrovirus-A (Perv A, SEQ ID NO:37), Porcine Endogeneous Retrovirus-B (Perv B, SEQ ID NO: 38), Human T Leukemia Virus-2 (HTLV2, SEQ ID NO:40) or Bovine Leukemia Virus (BLV, SEQ ID NO: 41).

In another advantageous embodiment, said at least one soluble receptor binding ligand is a set of two, three, four, five, six seven, eight or nine receptor binding ligands selected from the list consisting of: Amphotropic Murine Leukemia Retrovirus (AMLV, SEQ ID NO:32), Feline endogenous virus (RD114, SEQ ID NO:33), Xenotropic Murine Leukemia Virus (NZB, Xeno, SEQ ID NO: 34), Feline Leukemia Virus C (FeLVC, SEQ ID NO: 35), Koala Retrovirus (KoRV, SEQ ID NO: 36), Porcine Endogeneous Retrovirus-A (Perv A, SEQ ID NO:37), Porcine Endogeneous Retrovirus-B (Perv B, SEQ ID NO: 38), Human T Leukemia Virus-2 (HTLV2, SEQ ID NO:40) or Bovine Leukemia Virus (BLV, SEQ ID NO: 41), and allows the identification and the quantification of the expression of at least two membrane receptors present on the surface of single viable cells.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Xenotropic Murine Leukemia Virus (NZB, Xeno, SEQ ID NO: 34), Feline Leukemia Virus C (FeLVC, SEQ ID NO: 35), Koala Retrovirus (KoRV, SEQ ID NO: 36), Porcine Endogeneous Retrovirus-A (Perv A, SEQ ID NO:37), Porcine Endogeneous Retrovirus-B (Perv B, SEQ ID NO: 38), Human T Leukemia Virus-2 (HTLV2, SEQ ID NO:40) or Bovine Leukemia Virus (BLV, SEQ ID NO: 41), and wherein said at least one soluble receptor binding ligand is liable to interact with at least one membrane receptor of said single viable cells and wherein said membrane receptors are selected from the list consisting in PiT2, XPR1, PiT1, ASCT1, ASCT2, FLVCR1, RFT1, RFT3, Glut1.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Xenotropic Murine Leukemia Virus (NZB, Xeno, SEQ ID NO: 34), Feline Leukemia Virus C (FeLVC, SEQ ID NO: 35), Koala Retrovirus (KoRV, SEQ ID NO: 36), Porcine Endogeneous Retrovirus-A (Perv A, SEQ ID NO:37), Porcine Endogeneous Retrovirus-B (Perv B, SEQ ID NO: 38), Human T Leukemia Virus-2 (HTLV2, SEQ ID NO:40) or Bovine Leukemia Virus (BLV, SEQ ID NO: 41), and wherein said at least one soluble receptor binding ligand is liable to interact with at least one membrane receptor of said single viable cells and wherein said membrane receptors are selected from the list consisting in PiT2, XPR1, PiT1, ASCT1, ASCT2, FLVCR1, RFT1, RFT3, Glut1, and wherein the number of standard deviation (SD) between the sample tested and the sample mean for at least one receptor binding ligand is −1, preferably comprised between about −1 and about −10, in particular comprised between about −1 and about −3, being indicative of an underexpression of at least one membrane receptor allowing to diagnose a breast cancer.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Xenotropic Murine Leukemia Virus (NZB, Xeno, SEQ ID NO: 34), Feline Leukemia Virus C (FeLVC, SEQ ID NO: 35), Koala Retrovirus (KoRV, SEQ ID NO: 36), Porcine Endogeneous Retrovirus-A (Perv A, SEQ ID NO:37), Porcine Endogeneous Retrovirus-B (Perv B, SEQ ID NO: 38), Human T Leukemia Virus-2 (HTLV2, SEQ ID NO:40) or Bovine Leukemia Virus (BLV, SEQ ID NO: 41), and wherein said at least one soluble receptor binding ligand is liable to interact with at least one membrane receptor of said single viable cells and wherein said membrane receptors are selected from the list consisting in PiT2, XPR1, PiT1, ASCT1, ASCT2, FLVCR1, RFT1, RFT3, Glut1, and wherein the number of standard deviation (SD) between the sample tested and the sample mean for at least one receptor binding ligand is >+1, preferably comprised between about +1 and about +10, in particular comprised between about +1 and about +3, being indicative of an overexpression of at least one membrane receptor allowing to diagnose a malignant solid.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Xenotropic Murine Leukemia Virus (NZB, Xeno, SEQ ID NO: 34), Feline Leukemia Virus C (FeLVC, SEQ ID NO: 35), Koala Retrovirus (KoRV, SEQ ID NO: 36), Porcine Endogeneous Retrovirus-A (Perv A, SEQ ID NO:37), Porcine Endogeneous Retrovirus-B (Perv B, SEQ ID NO: 38), Human T Leukemia Virus-2 (HTLV2, SEQ ID NO:40) or Bovine Leukemia Virus (BLV, SEQ ID NO: 41), and wherein said at least one soluble receptor binding ligand is liable to interact with at least one membrane receptor of said single viable cells and wherein said membrane receptors are selected from the list consisting in PiT2, XPR1, PiT1, ASCT1, ASCT2, FLVCR1, RFT1, RFT3, Glut1, and wherein the number of standard deviation (SD) between the sample tested and the sample mean for at least one receptor binding ligand is −1, preferably comprised between about −1 and about −10, in particular comprised between about −1 and about −3, being indicative of an underexpression of at least one membrane receptor allowing to diagnose a breast cancer and wherein the number of standard deviation (SD) between the sample tested and the sample mean for at least one receptor binding ligand is >+1, preferably comprised between about +1 and about +10, in particular comprised between about +1 and about +3, being indicative of an overexpression of at least one receptor binding ligand allowing to diagnose a breast cancer.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Koala Retrovirus (KoRV, SEQ ID NO: 36) or Human T Leukemia Virus-2 (HTLV2, SEQ ID NO:40), and wherein said tumor is a human breast cancer tumor.

In an advantageous embodiment, the present invention relates to the use of single viable cells obtained from a recovery process of single viable cells from a solid tumor, in particular from a recovery process of single viable cells from a solid tumor defined above, wherein said tumor is a human breast cancer tumor and wherein said at least one receptor binding ligand is selected from the list consisting of: Amphotropic Murine Leukemia Retrovirus (AMLV, SEQ ID NO:32), Feline endogenous virus (RD114, SEQ ID NO:33), Xenotropic Murine Leukemia Virus (NZB, Xeno, SEQ ID NO: 34), Feline Leukemia Virus C (FeLVC, SEQ ID NO: 35), Koala Retrovirus (KoRV, SEQ ID NO: 36), Porcine Endogeneous Retrovirus-A (Perv A, SEQ ID NO:37), Porcine Endogeneous Retrovirus-B (Perv B, SEQ ID NO: 38), Human T Leukemia Virus-2 (HTLV2, SEQ ID NO:40) or Bovine Leukemia Virus (BLV, SEQ ID NO: 41), and wherein said tumor is a human breast cancer tumor selected from the group consisting of: HBCx-3, HBCx-4A, HBCx-8, HBCx-9, HBCx-10, HBCx-12A, HBCx-14, HBCx-22, HBCx-24, HBCx-30 and HBCx-41.

Thus, the quantification of the expression of membrane receptors in sample of a biological material previously excised from a patient suspected to have a solid breast cancer tumor, i.e. the evaluation of the overexpression and/or the underexpression and/or a median expression of at least one membrane receptor as determined by the level of expression of said receptor and found respectively significantly higher, lower or equal to the mean of the levels of expression of several samples of different breast cancer tumors allows to diagnostic the presence or not of a human breast cancer tumor in said sample, in particular of a specific breast cancer.

In an advantageous embodiment, the number of standard deviation (SD) between the sample tested and the sample mean for the receptor binding ligand RD114 (SEQ ID NO:33) is >+1, preferably comprised between about +1 and about +10, in particular comprised between about +1 and about +3, and is indicative of a HBCx-3 breast cancer.

In an advantageous embodiment, the number of standard deviation (SD) between the sample tested and the sample mean for the receptor binding ligand RD114 (SEQ ID NO:33) and/or PervB (SEQ ID NO: 38) and/or (KoRV, SEQ ID NO: 36) is (are)≦−1, preferably comprised between about −1 and about −10, in particular comprised between about −1 and about −3, and is (are) indicative of a HBCx-4A breast cancer.

In an advantageous embodiment, the number of standard deviation (SD) between the sample tested and the sample mean for the receptor binding ligand BLV (SEQ ID NO:41) and/or Xeno (SEQ ID NO: 34) is(are) ≧1, preferably comprised between about +1 and about +10, in particular comprised between about +1 and about +3, and is (are) indicative of a HBCx-8 breast cancer.

In an advantageous embodiment, the number of standard deviation (SD) between the sample tested and the sample mean for the receptor binding ligand Perv A (SEQ ID NO:37)≦−1, preferably comprised between about −1 and about −10, in particular comprised between about −1 and about −3, and is indicative of a HBCx-9 breast cancer.

In an advantageous embodiment, the number of standard deviation (SD) between the sample tested and the sample mean for the receptor binding ligand RD114 (SEQ ID NO:33) is <−1, preferably comprised between about −1 and about −10, in particular comprised between about −1 and about −3, and/or the number of standard deviation (SD) between the sample tested and the sample mean for the receptor binding ligand Xeno (SEQ ID NO: 34) is ≧1, preferably comprised between about +1 and about +10, in particular comprised between about +1 and about +3, and is indicative of a HBCx-24 breast cancer.

In an advantageous embodiment, the number of standard deviation (SD) between the sample tested and the sample mean for the receptor binding ligand AMLV (SEQ ID NO:32) and/or FeLVC (SEQ ID NO: 35) is (are) ≧1, preferably comprised between about +1 and about +10, in particular comprised between about +1 and about +3, and is (are) indicative of a HBCx-30 breast cancer. In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Koala Retrovirus (KoRV, SEQ ID NO: 36) or Human T Leukemia Virus-2 (HTLV2, SEQ ID NO:40).

In an advantageous embodiment, said at least one receptor binding ligand selected from the list consisting of: Amphotropic Murine Leukemia Retrovirus (AMLV, SEQ ID NO:32), Feline endogenous virus (RD114, SEQ ID NO:33), Koala Retrovirus (KoRV, SEQ ID NO: 36) or Human T Leukemia Virus-2 (HTLV2, SEQ ID NO:40) allows to diagnostic the presence or not of a breast cancer.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is a set of two receptor binding ligands selected from the list consisting of the following couple: (AMLV, SEQ ID NO:32) and (RD114, SEQ ID NO:33), (AMLV, SEQ ID NO:32) and (KoRV, SEQ ID NO: 36), (AMLV, SEQ ID NO:32) and (HTLV2, SEQ ID NO:40), (RD114, SEQ ID NO:33) and (KoRV, SEQ ID NO: 36), (RD114, SEQ ID NO:33) and (HTLV2, SEQ ID NO:40), (RD114, SEQ ID NO:33) and (HTLV2, SEQ ID NO:40).

In an advantageous embodiment, said set of two receptor binding ligands selected from the list consisting of the following couple: (AMLV, SEQ ID NO:32) and (RD114, SEQ ID NO:33), (AMLV, SEQ ID NO:32) and (KoRV, SEQ ID NO: 36), (AMLV, SEQ ID NO:32) and (HTLV2, SEQ ID NO:40), (RD114, SEQ ID NO:33) and (KoRV, SEQ ID NO: 36), (RD114, SEQ ID NO:33) and (HTLV2, SEQ ID NO:40), (RD114, SEQ ID NO:33) and (HTLV2, SEQ ID NO:40) allows to diagnostic the presence or not of a breast cancer. In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is a set of three receptor binding ligands selected from the list consisting of the following: (AMLV, SEQ ID NO:32) and (RD114, SEQ ID NO:33) and (KoRV, SEQ ID NO: 36), (AMLV, SEQ ID NO:32) and (RD114, SEQ ID NO:33) and (HTLV2, SEQ ID NO:40), (AMLV, SEQ ID NO:32) and (KoRV, SEQ ID NO: 36) and (HTLV2, SEQ ID NO:40), (RD114, SEQ ID NO:33) and (KoRV, SEQ ID NO: 36) and (HTLV2, SEQ ID NO:40).

In an advantageous embodiment, said set of three receptor binding ligands selected from the list consisting of the following: (AMLV, SEQ ID NO:32) and (RD114, SEQ ID NO:33) and (KoRV, SEQ ID NO: 36), (AMLV, SEQ ID NO:32) and (RD114, SEQ ID NO:33) and (HTLV2, SEQ ID NO:40), (AMLV, SEQ ID NO:32) and (KoRV, SEQ ID NO: 36) and (HTLV2, SEQ ID NO:40), (RD114, SEQ ID NO:33) and (KoRV, SEQ ID NO: 36) and (HTLV2, SEQ ID NO:40) allows to diagnostic the presence or not of a breast cancer. In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is a set of four receptor binding ligands consisting of: (AMLV, SEQ ID NO:32), (RD114, SEQ ID NO:33), (KoRV, SEQ ID NO: 36) and (HTLV2, SEQ ID NO:40).

In an advantageous embodiment, said set of four receptor binding ligands selected from the list consisting of the following: (AMLV, SEQ ID NO:32), (RD114, SEQ ID NO:33), (KoRV, SEQ ID NO: 36) and (HTLV2, SEQ ID NO:40) allows to diagnostic the presence or not of a breast cancer. In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Koala Retrovirus (KoRV, SEQ ID NO: 36) or Human T Leukemia Virus-2 (HTLV2, SEQ ID NO:40), and wherein said at least one soluble receptor binding ligand is liable to interact with at least one membrane receptor of said single viable cells and wherein said membrane receptors are selected from the list consisting in PiT2, PiT1, ASCT2, Glut1.

In an advantageous embodiment, said at least one receptor binding ligand and said at least one membrane receptor allows to diagnostic the presence or not of a breast cancer.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Koala Retrovirus (KoRV, SEQ ID NO: 36) or Human T Leukemia Virus-2 (HTLV2, SEQ ID NO:40), and wherein said tumor is a human breast cancer tumor selected from the group consisting of: HBCx-3, HBCx-4A, HBCx-8, HBCx-24, HBCx-30.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Koala Retrovirus (KoRV, SEQ ID NO: 36) or Human T Leukemia Virus-2 (HTLV2, SEQ ID NO:40), and wherein said at least one soluble receptor binding ligand is liable to interact with at least one membrane receptor of said single viable cells, wherein said membrane receptor is ASCT2, and said tumor is a HBCx-3 human breast cancer tumor, ASCT2 receptor being overexpressed.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Koala Retrovirus (KoRV, SEQ ID NO: 36) or Human T Leukemia Virus-2 (HTLV2, SEQ ID NO:40), and wherein said at least one soluble receptor binding ligand is liable to interact with at least one membrane receptor of said single viable cells, wherein said at least one membrane receptor is Glut1 and PiT1 and ASCT2, said tumor is a HBCx-4A or HBCx-24 human breast cancer tumor, and Glut1, PiT1 and ASCT2 receptors being underexpressed.

By the expression “Glut1, PiT1 and ASCT2 receptors being underexpressed”, it must be understood that the level of expression of said receptor is significantly lower than the mean of the levels of expression of several samples of different breast cancer tumors.

Thus, the quantification of the expression of Glut1, PiT1 and ASCT2 membrane receptors in sample of a biological material previously excised from a patient suspected to have a solid breast cancer tumor allows to diagnostic the presence or not of HBCx-4A or HBCx-24 human breast cancer tumor in said sample.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Koala Retrovirus (KoRV, SEQ ID NO: 36) or Human T Leukemia Virus-2 (HTLV2, SEQ ID NO:40), wherein said at least one soluble receptor binding ligand is liable to interact with at least one membrane receptor of said single viable cells, wherein said membrane receptor is PiT2, and said tumor is a HBCx-30 human breast cancer tumor, PiT2 receptor being overexpressed.

By the expression “PiT2 and receptors being overexpressed”, it must be understood that the level of expression of said receptor is significantly higher than the mean of the levels of expression of several samples of different breast cancer tumors.

Thus, the quantification of the expression of PiT2 membrane receptors in sample of a biological material previously excised from a patient suspected to have a solid breast cancer tumor allows to diagnostic the presence or not of HBCx-30 human breast cancer tumor in said sample.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Xenotropic Murine Leukemia Virus (NZB, Xeno, SEQ ID NO: 34), Feline Leukemia Virus C (FeLVC, SEQ ID NO: 35), Koala Retrovirus (KoRV, SEQ ID NO: 36), Porcine Endogeneous Retrovirus-A (Perv A, SEQ ID NO:37), Porcine Endogeneous Retrovirus-B (Perv B, SEQ ID NO: 38), Human T Leukemia Virus-2 (HTLV2, SEQ ID NO:40) or Bovine Leukemia Virus (BLV, SEQ ID NO: 41), and wherein said at least one soluble receptor binding ligand is liable to interact with at least one membrane receptor of said single viable cells and wherein said membrane receptors are selected from the list consisting in PiT2, XPR1, PiT1, ASCT1, ASCT2, FLVCR1, RFT1, RFT3, Glut1, and wherein said tumor is an UvMel melanoma.

In an advantageous embodiment, the present invention relates to the use of single viable cells obtained from a recovery process of single viable cells from a solid tumor, in particular from a recovery process of single viable cells from a solid tumor defined above, wherein said tumor is an UvMel melanoma and wherein said at least one receptor binding ligand is selected from the list consisting of: Amphotropic Murine Leukemia Retrovirus (AMLV, SEQ ID NO:32), Feline endogenous virus (RD114, SEQ ID NO:33), Xenotropic Murine Leukemia Virus (NZB, Xeno, SEQ ID NO: 34), Feline Leukemia Virus C (FeLVC, SEQ ID NO: 35), Koala Retrovirus (KoRV, SEQ ID NO: 36), Porcine Endogeneous Retrovirus-A (Perv A, SEQ ID NO:37), Porcine Endogeneous Retrovirus-B (Perv B, SEQ ID NO: 38), Human T Leukemia Virus-2 (HTLV2, SEQ ID NO:40) or Bovine Leukemia Virus (BLV, SEQ ID NO: 41), and wherein said tumor is an UvMel melanoma selected from the group consisting of: MP34, MP38, MP41, MP42, MP46, MP47, MP55, MP71, MP77, MP80, MM26, MM33, MM52, MP65, MM66 and MP74, in particular MM33, MP34, MP41, MP55.

Thus, the quantification of the expression of membrane receptors in sample of a biological material previously excised from a patient suspected to have an UvMel melanoma, i.e. the evaluation of the overexpression and/or the underexpression and/or a mean expression of at least one membrane receptor as determined by the level of expression of said receptor and found respectively significantly higher, lower or equal to the mean of the levels of expression of several samples of different UvMel melanoma allows to diagnostic the presence or not of a human UvMel melanoma in said sample, in particular of a specific UvMel melanoma.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is selected from the list consisting of: Amphotropic Murine Leukemia Retrovirus (AMLV, SEQ ID NO:32), Xenotropic Murine Leukemia Virus (NZB, Xeno, SEQ ID NO: 34), Porcine Endogeneous Retrovirus-A (Perv A, SEQ ID NO:37), Human T Leukemia Virus-2 (HTLV2, SEQ ID NO:40) or Bovine Leukemia Virus (BLV, SEQ ID NO: 41).

In an advantageous, said at least one receptor binding ligand is selected from the list consisting of: Amphotropic Murine Leukemia Retrovirus (AMLV, SEQ ID NO:32), Xenotropic Murine Leukemia Virus (NZB, Xeno, SEQ ID NO: 34), Porcine Endogeneous Retrovirus-A (Perv A, SEQ ID NO:37), Human T Leukemia Virus-2 (HTLV2, SEQ ID NO:40) or Bovine Leukemia Virus (BLV, SEQ ID NO: 41) allows to diagnostic the presence or not of an UvMel, such as MP34, MM33, MP41 or MP55.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is a set of two receptor binding ligands selected from the list consisting of the following couple: (AMLV, SEQ ID NO:32) and (Perv A, SEQ ID NO:37), (AMLV, SEQ ID NO:32) and (NZB, Xeno, SEQ ID NO: 34), (AMLV, SEQ ID NO:32) and (BLV, SEQ ID NO: 41), (AMLV, SEQ ID NO:32) and (HTLV2, SEQ ID NO:40), (Perv A, SEQ ID NO:37) and (NZB, Xeno, SEQ ID NO: 34), (Perv A, SEQ ID NO:37) and (BLV, SEQ ID NO: 41), (Perv A, SEQ ID NO:37) and (HTLV2, SEQ ID NO:40), (NZB, Xeno, SEQ ID NO: 34) and (BLV, SEQ ID NO: 41), (NZB, Xeno, SEQ ID NO: 34) and (HTLV2, SEQ ID NO:40), and (BLV, SEQ ID NO: 41) and (HTLV2, SEQ ID NO:40).

In an advantageous embodiment, said set of two receptor binding ligands selected from the list consisting of the following couple: (AMLV, SEQ ID NO:32) and (Perv A, SEQ ID NO:37), (AMLV, SEQ ID NO:32) and (NZB, Xeno, SEQ ID NO: 34), (AMLV, SEQ ID NO:32) and (BLV, SEQ ID NO: 41), (AMLV, SEQ ID NO:32) and (HTLV2, SEQ ID NO:40), (Perv A, SEQ ID NO:37) and (NZB, Xeno, SEQ ID NO: 34), (Perv A, SEQ ID NO:37) and (BLV, SEQ ID NO: 41), (Perv A, SEQ ID NO:37) and (HTLV2, SEQ ID NO:40), (NZB, Xeno, SEQ ID NO: 34) and (BLV, SEQ ID NO: 41), (NZB, Xeno, SEQ ID NO: 34) and (HTLV2, SEQ ID NO:40), and (BLV, SEQ ID NO: 41) and (HTLV2, SEQ ID NO:40) allows to diagnostic the presence or not of an UvMel, such as MP34, MM33, MP41 or MP55.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is a set of three receptor binding ligands selected from the list consisting of the following: (AMLV, SEQ ID NO:32) and (Perv A, SEQ ID NO:37) and (NZB, Xeno, SEQ ID NO: 34), (AMLV, SEQ ID NO:32) and (Perv A, SEQ ID NO:37) and (BLV, SEQ ID NO: 41), (AMLV, SEQ ID NO:32) and (Perv A, SEQ ID NO:37) and (HTLV2, SEQ ID NO:40), (AMLV, SEQ ID NO:32) and (NZB, Xeno, SEQ ID NO: 34) and (BLV, SEQ ID NO: 41), (AMLV, SEQ ID NO:32) and (NZB, Xeno, SEQ ID NO: 34) and (HTLV2, SEQ ID NO:40), (AMLV, SEQ ID NO:32) and (BLV, SEQ ID NO: 41), and (HTLV2, SEQ ID NO:40), (Perv A, SEQ ID NO:37) and (NZB, Xeno, SEQ ID NO: 34) and (BLV, SEQ ID NO: 41), (Perv A, SEQ ID NO:37) and (NZB, Xeno, SEQ ID NO: 34), and (HTLV2, SEQ ID NO:40), and NZB, Xeno, SEQ ID NO: 34) and (BLV, SEQ ID NO: 41), and (HTLV2, SEQ ID NO:40).

In an advantageous embodiment, said set of three receptor binding ligands selected from the list consisting of the following couple: (AMLV, SEQ ID NO:32) and (Perv A, SEQ ID NO:37) and (NZB, Xeno, SEQ ID NO: 34), (AMLV, SEQ ID NO:32) and (Perv A, SEQ ID NO:37) and (BLV, SEQ ID NO: 41), (AMLV, SEQ ID NO:32) and (Perv A, SEQ ID NO:37) and (HTLV2, SEQ ID NO:40), (AMLV, SEQ ID NO:32) and (NZB, Xeno, SEQ ID NO: 34) and (BLV, SEQ ID NO: 41), (AMLV, SEQ ID NO:32) and (NZB, Xeno, SEQ ID NO: 34) and (HTLV2, SEQ ID NO:40), (AMLV, SEQ ID NO:32) and (BLV, SEQ ID NO: 41), and (HTLV2, SEQ ID NO:40), (Perv A, SEQ ID NO:37) and (NZB, Xeno, SEQ ID NO: 34) and (BLV, SEQ ID NO: 41), (Perv A, SEQ ID NO:37) and (NZB, Xeno, SEQ ID NO: 34), and (HTLV2, SEQ ID NO:40), and NZB, Xeno, SEQ ID NO: 34) and (BLV, SEQ ID NO: 41), and (HTLV2, SEQ ID NO:40) allows to diagnostic the presence or not of an UvMel, such as MP34, MM33, MP41 or MP55.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is a set of four receptor binding ligands consisting of: (AMLV, SEQ ID NO:32) and (Perv A, SEQ ID NO:37) and (NZB, Xeno, SEQ ID NO: 34) and (BLV, SEQ ID NO: 41), (AMLV, SEQ ID NO:32) and (Perv A, SEQ ID NO:37) and (NZB, Xeno, SEQ ID NO: 34) and (HTLV2, SEQ ID NO:40),

### (AMLV, SEQ ID NO:32) and (NZB, Xeno, SEQ ID NO: 34) and (BLV, SEQ ID NO: 41) and (HTLV2, SEQ ID NO:40), and (Perv A, SEQ ID NO:37) and (NZB, Xeno, SEQ ID NO: 34) and (BLV, SEQ ID NO: 41) and (HTLV2, SEQ ID NO:40).

In an advantageous embodiment, said set of four receptor binding ligands selected from the list consisting of the following couple: (AMLV, SEQ ID NO:32) and (Perv A, SEQ ID NO:37) and (NZB, Xeno, SEQ ID NO: 34) and (BLV, SEQ ID NO: 41), (AMLV, SEQ ID NO:32) and (Perv A, SEQ ID NO:37) and (NZB, Xeno, SEQ ID NO: 34) and (HTLV2, SEQ ID NO:40),

(AMLV, SEQ ID NO:32) and (NZB, Xeno, SEQ ID NO: 34) and (BLV, SEQ ID NO: 41) and (HTLV2, SEQ ID NO:40), and (Perv A, SEQ ID NO:37) and (NZB, Xeno, SEQ ID NO: 34) and (BLV, SEQ ID NO: 41) and (HTLV2, SEQ ID NO:40) allows to diagnostic the presence or not of an UvMel, such as MP34, MM33, MP41 or MP55.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is selected from the list consisting of: Amphotropic Murine Leukemia Retrovirus (AMLV, SEQ ID NO:32), Xenotropic Murine Leukemia Virus (NZB, Xeno, SEQ ID NO: 34), Porcine Endogeneous Retrovirus-A (Perv A, SEQ ID NO:37), Human T Leukemia Virus-2 (HTLV2, SEQ ID NO:40) or Bovine Leukemia Virus (BLV, SEQ ID NO: 41), and wherein said at least one soluble receptor binding ligand is liable to interact with at least one membrane receptor of said single viable cells and wherein said membrane receptors are selected from the list consisting in PiT2, RFT3, RFT1, XPR1 and Glut1.

In an advantageous embodiment, said at least one receptor binding ligand and said at least one membrane receptor allows to diagnostic the presence or not of an UvMel, such as MP34, MM33, MP41 or MP55.

In an advantageous embodiment, the present invention relates to the use of single viable cells obtained from a recovery process of single viable cells from a solid tumor, in particular from a recovery process of single viable cells from a solid tumor defined above, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Xenotropic Murine Leukemia Virus (NZB, Xeno, SEQ ID NO: 34), Feline Leukemia Virus C (FeLVC, SEQ ID NO: 35), Koala Retrovirus (KoRV, SEQ ID NO: 36), Porcine Endogeneous Retrovirus-A (Perv A, SEQ ID NO:37), Porcine Endogeneous Retrovirus-B (Perv B, SEQ ID NO: 38), Human T Leukemia Virus-2 (HTLV2, SEQ ID NO:40) or Bovine Leukemia Virus (BLV, SEQ ID NO: 41) to discriminate the presence or not of a human UvMel melanoma versus a breast cancer.

Thus, in this embodiment, the quantification of the expression of membrane receptors in sample of a biological material previously excised from a patient suspected to have solid tumor, i.e. the evaluation of the overexpression and/or the underexpression and/or a median expression of at least one membrane receptor as determined by the level of expression of said receptor and found respectively significantly higher, lower or equal to the mean of the levels of expression of several samples of different solid tumor allows to discriminate the presence or not of a human UvMel melanoma versus a breast cancer in said sample.

In an advantageous embodiment, the present invention relates to the use of single viable cells obtained from a recovery process of single viable cells from a solid tumor, in particular from a recovery process of single viable cells from a solid tumor to discriminate the presence or not of a human UvMel melanoma versus a breast cancer, defined above, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Feline Leukemia Virus C (FeLVC, SEQ ID NO: 35), Porcine Endogeneous Retrovirus-A (Perv A, SEQ ID NO:37), Human T Leukemia Virus-2 (HTLV2, SEQ ID NO:40) or Bovine Leukemia Virus (BLV, SEQ ID NO: 41).

In an advantageous embodiment, the receptor binding to Feline Leukemia Virus C (FeLVC, SEQ ID NO: 35) is overexpressed in UvMel compared with breast cancer, and/or the receptor binding to Amphotropic Murine Leukemia Retrovirus (AMLV, SEQ ID NO:32) is underexpressed in UvMel compared with breast cancer, and/or the receptor binding to Porcine Endogeneous Retrovirus-A (Perv A, SEQ ID NO:37) is underexpressed in UvMel compared with breast cancer, and/or the receptor binding to Feline endogenous virus (RD114, SEQ ID NO:33) is overexpressed in UvMel compared with breast cancer, and/or the receptor binding to Bovine Leukemia Virus (BLV, SEQ ID NO: 41) overexpressed in UvMel compared with breast cancer, and/or the receptor binding to Human T Leukemia Virus-2 (HTLV2, SEQ ID NO:40) overexpressed in UvMel compared with breast cancer.

Therefore, RBDs can discriminate different types of solid tumor, in particular UvMel and breast cancer allowing to have a specific signature of tumor and/or cellular type.

In an advantageous embodiment, the present invention relates to the use of single viable cells obtained from a recovery process of single viable cells from a solid tumor, in particular from a recovery process of single viable cells from a solid tumor to discriminate the presence or not of a human UvMel melanoma versus a breast cancer, defined above, wherein said at least one receptor binding ligand is a set of two receptor binding ligands selected from the list consisting of: (FeLVC, SEQ ID NO: 35) and (AMLV, SEQ ID NO:32), (FeLVC, SEQ ID NO: 35) and (Perv A, SEQ ID NO:37), (FeLVC, SEQ ID NO: 35) and (RD114, SEQ ID NO:33), (FeLVC, SEQ ID NO: 35) and (BLV, SEQ ID NO: 41), (FeLVC, SEQ ID NO: 35) and (HTLV2, SEQ ID NO:40), (AMLV, SEQ ID NO:32) and (Perv A, SEQ ID NO:37), (AMLV, SEQ ID NO:32) and (RD114, SEQ ID NO:33), (AMLV, SEQ ID NO:32) and (BLV, SEQ ID NO: 41), (AMLV, SEQ ID NO:32) and (HTLV2, SEQ ID NO:40), (Perv A, SEQ ID NO:37) and (RD114, SEQ ID NO:33), (Perv A, SEQ ID NO:37) and (BLV, SEQ ID NO: 41), (Perv A, SEQ ID NO:37) and (HTLV2, SEQ ID NO:40), (RD114, SEQ ID NO:33) and (BLV, SEQ ID NO: 41), (RD114, SEQ ID NO:33) and (HTLV2, SEQ ID NO:40), (BLV, SEQ ID NO: 41) and (HTLV2, SEQ ID NO:40), in particular a set of two receptor binding ligands selected from the list consisting of the couples: (FeLVC, SEQ ID NO: 35) and (Perv A, SEQ ID NO:37), (FeLVC, SEQ ID NO: 35) and (BLV, SEQ ID NO: 41), (FeLVC, SEQ ID NO: 35) and (HTLV2, SEQ ID NO:40), (Perv A, SEQ ID NO:37) and (BLV, SEQ ID NO: 41), (Perv A, SEQ ID NO:37) and (HTLV2, SEQ ID NO:40), (BLV, SEQ ID NO: 41) and (HTLV2, SEQ ID NO:40).

The detection of two receptors binding to said couple RBDs and having an overexpression or underexpression as defined above, allows a better diagnosis of UvMel or breast cancer compared with the detection of one receptor.

In an advantageous embodiment, the present invention relates to the use of single viable cells obtained from a recovery process of single viable cells from a solid tumor, in particular from a recovery process of single viable cells from a solid tumor to discriminate the presence or not of a human UvMel melanoma versus a breast cancer, defined above, wherein said at least one receptor binding ligand is a set of three receptor binding ligands selected from the list consisting of: (FeLVC, SEQ ID NO: 35) and (AMLV, SEQ ID NO:32) and (Perv A, SEQ ID NO:37), (FeLVC, SEQ ID NO: 35) and (AMLV, SEQ ID NO:32) and (RD114, SEQ ID NO:33), (FeLVC, SEQ ID NO: 35) and (AMLV, SEQ ID NO:32) and (BLV, SEQ ID NO: 41), (FeLVC, SEQ ID NO: 35) and (AMLV, SEQ ID NO:32) and (HTLV2, SEQ ID NO:40), (FeLVC, SEQ ID NO: 35) and (Perv A, SEQ ID NO:37) and (RD114, SEQ ID NO:33), (FeLVC, SEQ ID NO: 35) and (Perv A, SEQ ID NO:37) and (BLV, SEQ ID NO: 41), (FeLVC, SEQ ID NO: 35) and (Perv A, SEQ ID NO:37) and (HTLV2, SEQ ID NO:40), (FeLVC, SEQ ID NO: 35) and (RD114, SEQ ID NO:33) and (BLV, SEQ ID NO: 41), (FeLVC, SEQ ID NO: 35) and (RD114, SEQ ID NO:33) and (HTLV2, SEQ ID NO:40), (FeLVC, SEQ ID NO: 35) and (BLV, SEQ ID NO: 41) and (HTLV2, SEQ ID NO:40), (AMLV, SEQ ID NO:32) and (Perv A, SEQ ID NO:37) and (RD114, SEQ ID NO:33), (AMLV, SEQ ID NO:32) and (Perv A, SEQ ID NO:37) and (BLV, SEQ ID NO: 41), (AMLV, SEQ ID NO:32) and (Perv A, SEQ ID NO:37) and (HTLV2, SEQ ID NO:40), (Perv A, SEQ ID NO:37) and (RD114, SEQ ID NO:33) and (BLV, SEQ ID NO: 41), (Perv A, SEQ ID NO:37) and (RD114, SEQ ID NO:33) and (HTLV2, SEQ ID NO:40), (Perv A, SEQ ID NO:37) and (BLV, SEQ ID NO: 41) and (HTLV2, SEQ ID NO:40), (RD114, SEQ ID NO:33) and (BLV, SEQ ID NO: 41) and (HTLV2, SEQ ID NO:40), in particular a set of two receptor binding ligands selected from the list consisting of: (FeLVC, SEQ ID NO: 35) and (Perv A, SEQ ID NO:37) and (BLV, SEQ ID NO: 41), (FeLVC, SEQ ID NO: 35) and (Perv A, SEQ ID NO:37) and (HTLV2, SEQ ID NO:40), (FeLVC, SEQ ID NO: 35) and (BLV, SEQ ID NO: 41) and (HTLV2, SEQ ID NO:40), (Perv A, SEQ ID NO:37) and (BLV, SEQ ID NO: 41) and (HTLV2, SEQ ID NO:40).

The detection of three receptors binding to said set of three RBDs and having an overexpression or underexpression as defined above, allows a better diagnosis of UvMel or breast cancer compared with the detection of two or one receptor.

In an advantageous embodiment, the present invention relates to the use of single viable cells obtained from a recovery process of single viable cells from a solid tumor, in particular from a recovery process of single viable cells from a solid tumor to discriminate the presence or not of a human UvMel melanoma versus a breast cancer, defined above, wherein said at least one receptor binding ligand is a set of four receptor binding ligands selected from the list consisting of: (FeLVC, SEQ ID NO: 35) and (AMLV, SEQ ID NO:32) and (Perv A, SEQ ID NO:37) and (RD114, SEQ ID NO:33), (FeLVC, SEQ ID NO: 35) and (AMLV, SEQ ID NO:32) and (Perv A, SEQ ID NO:37) and (BLV, SEQ ID NO: 41), (FeLVC, SEQ ID NO: 35) and (AMLV, SEQ ID NO:32) and (Perv A, SEQ ID NO:37) and (HTLV2, SEQ ID NO:40), (FeLVC, SEQ ID NO: 35) and (Perv A, SEQ ID NO:37) and (RD114, SEQ ID NO:33) and (BLV, SEQ ID NO: 41), (FeLVC, SEQ ID NO: 35) and (Perv A, SEQ ID NO:37) and (BLV, SEQ ID NO: 41) and (HTLV2, SEQ ID NO:40), (FeLVC, SEQ ID NO: 35) and (RD114, SEQ ID NO:33) and (BLV, SEQ ID NO: 41) and (HTLV2, SEQ ID NO:40), (AMLV, SEQ ID NO:32) and (Perv A, SEQ ID NO:37) and (RD114, SEQ ID NO:33) and (BLV, SEQ ID NO: 41), (AMLV, SEQ ID NO:32) and (Perv A, SEQ ID NO:37) and (RD114, SEQ ID NO:33) and (HTLV2, SEQ ID NO:40), (Perv A, SEQ ID NO:37) and (RD114, SEQ ID NO:33) and (BLV, SEQ ID NO: 41) and (HTLV2, SEQ ID NO:40), in particular (FeLVC, SEQ ID NO: 35) and (Perv A, SEQ ID NO:37) and (BLV, SEQ ID NO: 41) and (HTLV2, SEQ ID NO:40).

The detection of four receptors binding to said set of four RBDs and having an overexpression or underexpression as defined above, allows a better diagnosis of UvMel or breast cancer compared with the detection of three or two or one receptor.

In an advantageous embodiment, the present invention relates to the use of single viable cells obtained from a recovery process of single viable cells from a solid tumor, in particular from a recovery process of single viable cells from a solid tumor to discriminate the presence or not of a human UvMel melanoma versus a breast cancer, defined above, wherein said at least one receptor binding ligand is a set of five receptor binding ligands selected from the list consisting of: (FeLVC, SEQ ID NO: 35) and (AMLV, SEQ ID NO:32) and (Perv A, SEQ ID NO:37) and (RD114, SEQ ID NO:33) and (BLV, SEQ ID NO: 41), (FeLVC, SEQ ID NO: 35) and (AMLV, SEQ ID NO:32) and (Perv A, SEQ ID NO:37) and (RD114, SEQ ID NO:33) and (HTLV2, SEQ ID NO:40), (FeLVC, SEQ ID NO: 35) and (Perv A, SEQ ID NO:37) and (RD114, SEQ ID NO:33) and (BLV, SEQ ID NO: 41) and (HTLV2, SEQ ID NO:40).

The detection of five receptors binding to said set of five RBDs and having an overexpression or underexpression as defined above, allows a better diagnosis of UvMel or breast cancer compared with the detection of four or three or two or one receptor.

In an advantageous embodiment, the present invention relates to the use of single viable cells obtained from a recovery process of single viable cells from a solid tumor, in particular from a recovery process of single viable cells from a solid tumor to discriminate the presence or not of a human UvMel melanoma versus a breast cancer, defined above, wherein said at least one receptor binding ligand is a set of six receptor binding ligands selected from the list consisting of: (FeLVC, SEQ ID NO: 35) and (AMLV, SEQ ID NO:32) and (Perv A, SEQ ID NO:37) and (RD114, SEQ ID NO:33) and (BLV, SEQ ID NO: 41) and (HTLV2, SEQ ID NO:40).

The detection of six receptors binding to said set of six RBDs and having an overexpression or underexpression as defined above, allows a better diagnosis of UvMel or breast cancer compared with the detection of four or three or two or one receptor.

In an advantageous embodiment, the present invention relates to the use of single viable cells obtained from a recovery process of single viable cells from a solid tumor, in particular from a recovery process of single viable cells from a solid tumor to discriminate the presence or not of a human UvMel melanoma versus a breast cancer, defined above, wherein said at least one receptor binding ligand is a set of two receptor binding ligands selected from the list consisting of: (FeLVC, SEQ ID NO: 35) and (AMLV, SEQ ID NO:32), (FeLVC, SEQ ID NO: 35) and (Perv A, SEQ ID NO:37), (FeLVC, SEQ ID NO: 35) and (RD114, SEQ ID NO:33), (FeLVC, SEQ ID NO: 35) and (BLV, SEQ ID NO: 41), (FeLVC, SEQ ID NO: 35) and (HTLV2, SEQ ID NO:40), (AMLV, SEQ ID NO:32) and (Perv A, SEQ ID NO:37), (AMLV, SEQ ID NO:32) and (RD114, SEQ ID NO:33), (AMLV, SEQ ID NO:32) and (BLV, SEQ ID NO: 41), (AMLV, SEQ ID NO:32) and (HTLV2, SEQ ID NO:40), (Perv A, SEQ ID NO:37) and (RD114, SEQ ID NO:33), (Perv A, SEQ ID NO:37) and (BLV, SEQ ID NO: 41), (Perv A, SEQ ID NO:37) and (HTLV2, SEQ ID NO:40), (RD114, SEQ ID NO:33) and (BLV, SEQ ID NO: 41), (RD114, SEQ ID NO:33) and (HTLV2, SEQ ID NO:40), (BLV, SEQ ID NO: 41) and (HTLV2, SEQ ID NO:40), in particular a set of two receptor binding ligands selected from the list consisting of the couples: (FeLVC, SEQ ID NO: 35) and (Perv A, SEQ ID NO:37), (FeLVC, SEQ ID NO: 35) and (BLV, SEQ ID NO: 41), (FeLVC, SEQ ID NO: 35) and (HTLV2, SEQ ID NO:40), (Perv A, SEQ ID NO:37) and (BLV, SEQ ID NO: 41), (Perv A, SEQ ID NO:37) and (HTLV2, SEQ ID NO:40), (BLV, SEQ ID NO: 41) and (HTLV2, SEQ ID NO:40) and wherein said at least one soluble receptor binding ligand is liable to interact with at least one membrane receptor of said single viable cells and wherein said membrane receptors are selected from the list consisting in PiT2, XPR1, PiT1, ASCT1, ASCT2, FLVCR1, RFT1, RFT3, Glut1. In another aspect, the present invention relates to a process of diagnostic or prognostic of solid cancer tumors in a patient comprising:
a. Sampling of a biological material suspected to be a solid cancer tumor from a patient,
b. Optionally, grafting said tumor in a mouse,
c. Implementation of a recovery process of single viable cells as defined above, from a human solid tumor of step a, or a human solid tumor from mouse of step b after excision from the mouse,
d. Contacting said single viable cells from a human solid tumor from a patient or a human solid tumor from mouse of step c, with at least one soluble receptor binding ligands derived from the soluble part of the glycoprotein of an enveloped virus that interacts with a cellular cognate receptor,
e. identifying and quantifying the expression of membrane receptors present on the surface of said single viable cells of step d,
f. comparing the expression of membrane receptors obtained in each said single viable cells of step e with a respective control,
g. an overexpression and/or a underexpression of membrane receptors in each said single viable cells compared to their respective control being indicative of a solid cancer tumor.

In said process, the sampling of a biological material suspected to be a solid cancer tumor from a patient can be a biopsy.

In step d. the solid cancer tumor from the patient is then directly analyzed or the solid cancer tumor has previously been grafted in a mouse in step b. in order to have a clinical model available as a detecting tool of said solid cancer tumor or for further studies.

In step c., the recovery process of the single viable cells of the invention is used but a recovery process of single viable cells described in the prior art can also be used.

In step f, the control represents the sample mean for solid cancer tumor of the same type and for a given receptor binding ligand. The comparison of the expression of membranes receptors is made with the aid of said receptor binding ligands by means of the number of standard deviation (SD) between the sample tested and the sample mean as described above.

In step g, the overexpression of at least one membrane receptor and/or the underexpression of at least one membrane receptor allows to diagnose a cancer tumor and further to identify the cancer tumor type.

In an advantageous embodiment, the cancer tumor type diagnosed in the process above defined is a breast cancer tumor.

In another aspect, the present invention relates to a process of a therapeutic response assessment in a patient having a treatment against solid cancer tumors comprising:
a. Sampling of a tumor to be analyzed from a patient suffering from a solid cancer tumor and treated with an anticancer drug,
b. Optionally, grafting said tumor in a mouse,
c. Implementation of a recovery process of single viable cells defined above, from a human solid tumor of step a, or a human solid tumor from mouse of step b after excision from the mouse,
d. Contacting said single viable cells from a human solid tumor from a patient or a human solid tumor from mouse of step c, with at least one soluble receptor binding ligands derived from the soluble part of the glycoprotein of an enveloped virus that interacts with a cellular cognate receptor,
e. identifying and quantifying the expression of membrane receptors present on the surface of said single viable cells of step d,
f. comparing the expression of membrane receptors obtained in step e with the one obtained before treatment of said patient or said mouse,
g. an increase of the expression of an underexpressed membrane receptor or a decrease of an overexpressed membrane receptor obtained in each said single viable cells of step e compared respectively to the one obtained before treatment being respectively indicative of a therapeutic response by the patient or the mouse to a anticancer treatment.

In step c., the recovery process of the single viable cells of the invention is used but a recovery process of single viable cells described in the prior art can also be used.

The process of a therapeutic response assessment described here is very similar to the one used for the diagnosis.

It differs only with the use of a different control as the patient (or the mouse) before the treatment is a control in himself.

This process allows thus rapidly defining the efficacy of a cancer treatment and changing said treatment in the case where the patient is not responding enough or at all to said treatment.

It allows also determining, every year for example after the end of the treatment, an apparition again of said solid cancer tumor.

In an advantageous embodiment, the cancer tumor type in the process of therapeutic response assessment above defined is a breast cancer tumor.

In another aspect, the present invention relates to a screening process of a drug liable to treat a solid cancer tumor comprising:
a. Sampling of a tumor to be analyzed from a patient suffering from a solid cancer tumor,
b. Optionally, grafting said tumor in a mouse, and treating the mouse with a drug to test,
c. Implementation of a recovery process of single viable cells defined above, from a human solid tumor of step a, or a human solid tumor from a mouse of step b after excision from the mouse,
d. Culturing said single viable cells from a human solid tumor of step c and treating them with a drug to test,
e. Contacting said single viable cells from a human solid tumor from a patient of step d or a human solid tumor from mouse of step c, with at least one soluble receptor binding ligands derived from the soluble part of the glycoprotein of an enveloped virus that interacts with a cellular cognate receptor,
f. identifying and quantifying the expression of membrane receptors present on the surface of said single viable cells of step e,
g. comparing the expression of membrane receptors obtained in step f with the one obtained before treatment of said single viable cells from a human solid tumor or from human solid tumor from a mouse,
h. an increase of the expression of an underexpressed membrane receptor or a decrease of an overexpressed membrane receptor obtained in each said single viable cells of step f compared respectively to the one obtained before treatment being respectively indicative of a drug liable to treat a solid tumor.

In step c., the recovery process of the single viable cells of the invention is used but a recovery process of single viable cells described in the prior art can also be used.

The screening process described here is very similar to the one used for the diagnosis.

It differs only with the culturing of said single viable cells.

Said screening process allows thus to determine the existing molecule type that is the best and/or that is selective for a cancer cell compared to other cancer cell types for treating said solid cancer tumor but it also allows finding new molecules active and/or specific against a determined solid cancer tumor.

In an advantageous embodiment, the cancer tumor type of the screening process above defined is a breast cancer tumor.

In another aspect, the present invention relates to a process of diagnostic or prognostic of solid cancer tumors in a patient comprising:
a. Implementation of a recovery process of single viable cells from a solid tumor defined above, said solid tumor being a sample of a biological material suspected to be a solid cancer tumor:
   i. previously excised from a patient, or
   ii. previously excised from a patient, grafted in a mouse and then excised from said mouse,
b. Contacting said single viable cells of step a.i. or a.ii. with at least one soluble receptor binding ligands derived from the soluble part of the glycoprotein of an enveloped virus that interacts with a cellular cognate receptor,
c. identifying and quantifying the expression of membrane receptors present on the surface of said single viable cells contacted with at least one said soluble receptor binding ligands of step b,
d. comparing the expression of membrane receptors of each said single viable cells contacted with at least one said soluble receptor binding ligands obtained in step c with a respective control,
e. an overexpression and/or a underexpression of membrane receptors of each said single viable cells contacted with at least one said soluble receptor binding ligands obtained in step c, compared to their respective control, being indicative of a solid cancer tumor.

In step a., the recovery process of the single viable cells of the invention is used but a recovery process of single viable cells described in the prior art can also be used.

The process of diagnostic or prognostic described here is very similar to the process of diagnostic or prognostic described above.

The only difference is that the tumor has previously been excised from a patient, and that the previously excised tumor from a patient has previously been grafted in a mouse and that the previously grated tumor has been previously excised from the mouse before carrying out said process.

In an advantageous embodiment, the cancer tumor type of the screening process above defined is a breast cancer tumor.

In an advantageous embodiment, the present invention relates to a process of a therapeutic response assessment in a patient having a treatment against solid cancer tumors comprising:
a. Implementation of a recovery process of single viable cells from a solid tumor defined above, said solid tumor being a sample of a tumor to be analyzed:
   iii. previously excised from a patient suffering from a solid cancer tumor and treated with an anticancer drug, or
   iv. previously:
      excised from a patient suffering from a solid tumor, grafted in a mouse that is then treated with a drug to test, said grafted tumor of said treated mouse being previously excised from said mouse,
b. Contacting said single viable cells of step a.i. or a.ii. with at least one soluble receptor binding ligands derived from the soluble part of the glycoprotein of an enveloped virus that interacts with a cellular cognate receptor,
c. identifying and quantifying the expression of membrane receptors present on the surface of said single viable cells, contacted with at least one said soluble receptor binding ligands of step b,
d. comparing the expression of membrane receptors present on the surface of each said single viable cells obtained in step c with the one obtained before treatment of said patient or said mouse,
e. an increase of the expression of an underexpressed membrane receptor or a decrease of an overexpressed membrane receptor of each said single viable cells contacted with at least one said soluble receptor binding ligands obtained in step c, compared with respectively the one obtained without treatment being indicative of a therapeutic response by the patient or the mouse to a anticancer treatment.

In step a., the recovery process of the single viable cells of the invention is used but a recovery process of single viable cells described in the prior art can also be used.

The process of a therapeutic response assessment described here is very similar to the process of a therapeutic response assessment described above.

The only difference is that the tumor has previously been excised from a patient, and that the previously excised tumor from a patient has previously been grafted in a mouse and that the previously grated tumor has been previously excised from the mouse before carrying out said process.

In an advantageous embodiment, the cancer tumor type of the screening process above defined is a breast cancer tumor.

In an advantageous embodiment, the present invention relates to a screening process of a drug liable to treat a solid cancer tumor comprising:
a. Implementation of a recovery process of single viable cells from a solid tumor defined above, said solid tumor being a sample of a tumor to be analyzed:
   v. previously excised from a patient suffering from a solid cancer tumor, or
   vi. previously:
      excised from a patient suffering from a solid tumor, grafted in a mouse and previously excised from said mouse,
b. Culturing said single viable cells of step a.i. and a.ii. and treating them with a drug to test,
c. Contacting said each treated single viable cells of step b. with at least one soluble receptor binding ligands derived from the soluble part of the glycoprotein of an enveloped virus that interacts with a cellular cognate receptor,
d. identifying and quantifying the expression of membrane receptors present on the surface of said each single viable cells,
e. comparing the expression of membrane receptors present on the surface of said each single viable cells obtained in step d with the one obtained without treatment of said each single viable cells,
f. an increase of the expression of an underexpressed membrane receptor or a decrease of an overexpressed membrane receptor present on the surface of said each single viable cells obtained in step d compared with the one obtained without treatment being indicative of a drug liable to treat a solid cancer tumor.

In step a., the recovery process of the single viable cells of the invention is used but a recovery process of single viable cells described in the prior art can also be used.

The screening process described here is very similar to the screening process described above.

The only difference is that the tumor has previously been excised from a patient, and that the previously excised tumor from a patient has previously been grafted in a mouse and that the previously grafted tumor has been previously excised from the mouse before carrying out said process.

In an advantageous embodiment, the cancer tumor type of the screening process above defined is a breast cancer tumor.

### DESCRIPTION OF THE FIGURES

The following figures and examples illustrate the invention.

FIG. 1 corresponds to the schematic representation of the mature Env protein of HTLV-1 (as a prototypic deltaretrovirus Env) and common motifs in the SU with Friend-MLV (as a prototypic gammaretrovirus Env). TM corresponds to the transmembrane domain and SU corresponds to the surface domain. RBD corresponds to the domain of SU that interacts with the membrane receptor of the target cell.

FIG. 2 presents the reproducibility of the optimized recovery process of the invention.

Recovery yields of viable cells for 8 different human breast cancer xenografts are shown (from left to right HBCx-3, HBCx-9, HBCx-10, HBCx-12A, HBCx-14, HBCx-22, HBCx-24, HBCx-41).

Dots represent the values for independent experiments and drawbars represent the mean.

FIG. 3A to 3E present the gating strategy for live human cells analysis or sorting.

FIG. 3A: Cells are selected based on on forward and side scatter.

FIG. 3B: and FIG. 3C) Aggregates are excluded using forward and side scatter area vs width.

FIG. 3D: Dead cells (DAPI positive) are excluded

FIG. 3E: pan mouse H2 negative, human EpCAM positive tumor cells are analysed for CD44 or RBD levels.

FIG. 4A to 4E present the comparison of FC and IHC analyses of CD44 expression.

FIG. 4A: Linear regression curve and coefficient of determination (R²) of CD44 positive cells by flow cytometric analysis of dissociated cells vs IHC analysis of tumor sections. FIG. 4B and FIG. 4C: Flow cytometric (left) vs IHC (right) analysis of (FIG. 4B) CD44 low and (FIG. 4C) CD44 high breast cancer xenografts (respectively HBCx-3 and HBCx-4A). Flow cytometric histograms of CD44 APCH7 vs EpCAM PerCPCY5.5. show the FMO control on the left and CD44 stained cells on the right. IHC slides (FIGS. 4D and 4E) are shown at high magnification (bar=20 μm).

FIG. 5 presents the surface expression of metabolite transporters (membrane receptors) of five breast cancer xenograft models. The mean expression level of each transporter is plotted in terms of MESF (Molecular Equivalent Soluble Fluorophore, see examples). Reproducibility was assessed by dissociating and labelling distinct tumors for each model in one, two or three independent experiments over time (p value was calculated using a Kruskal-Wallis test).

x-axis: metabolite transporters (membrane receptors), from left to right: Glut1, PiT1, PiT2 and ASCT2. For each metabolite transporter, from left to right: HBCx-3, HBCx-4A, HBCx-8, HBCx-24, HBCx-30.

y-axis: MESF (molecule of equivalent fluorophore).

FIG. 6A to 6C present the profiles obtained with ex vivo culture of dissociated cells.

FIG. 6A: Less than 12% of viable (DAPI negative) cells are apoptotic using an activated caspase 3 assay.

FIG. 6B: proliferation of dissociated cells cultured for at least 13 days as analysed by WST assay (circle: HBCx-9; hexagone: HBCx-9, triangle: HBCx-41 and diamond: HBCx-22).

HBCx-9 has been tested twice.

x-axis: time (days), y-axis: DO 450 nm.

FIG. 6C: Treatment with docetaxel black circle) or cisplatin (white square) of HBCx-41 in vitro cultured dissociated cells shows dose dependent toxicity similar to commonly used established cell lines.

x-axes: concentration of docetaxel black circle) or cisplatin (white square) (μM), y-axis cell viability (%)

FIG. 7 presents the cluster analysis of 6 distinct human breast cancer models (HBCx-3, HBCx-4A, HBCx-8, HBCx-9, HBCx-24, HBCx-30), grafted into mice.

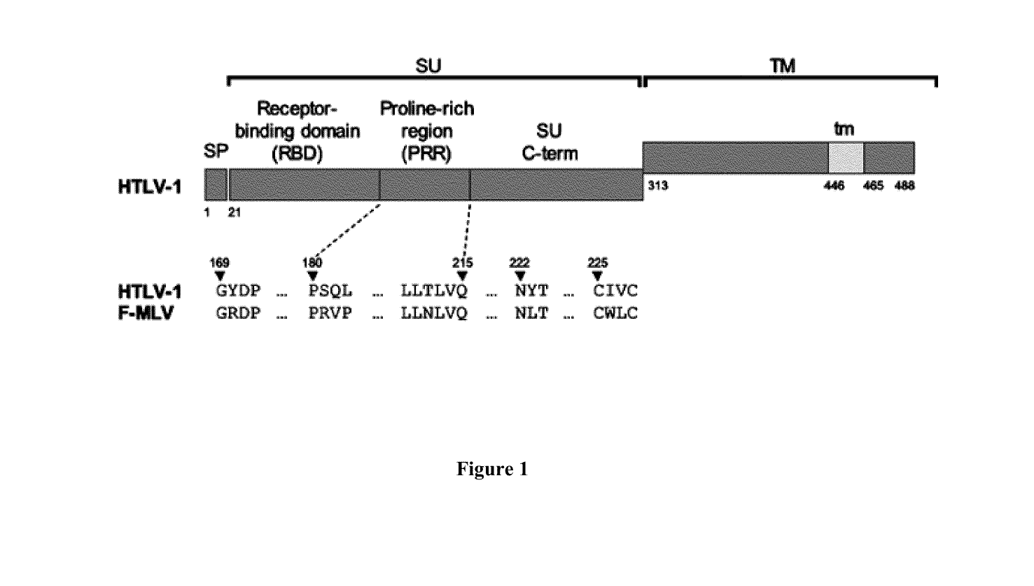

10 metabolite transporters (membrane receptors) were quantified through the use of 10 receptor binding ligand (SEQ ID NO: 32-41) fused to either mouse or rabbit IgG Fc fragments (mFC and rFC, respectively), giving rise to the multiplexed signature, which revealed to be specific of each HBCx model. All signatures were reproducibly obtained through comparison of distinct tumors belonging to each model, along different experiments.

Mean represent the sample mean of the absolute values obtained for one receptor binding ligand with the 6 distinct human breast cancer models.

SD represents the standard deviation.

The number of SD represents the number of SD separating the sample X from the sample mean.

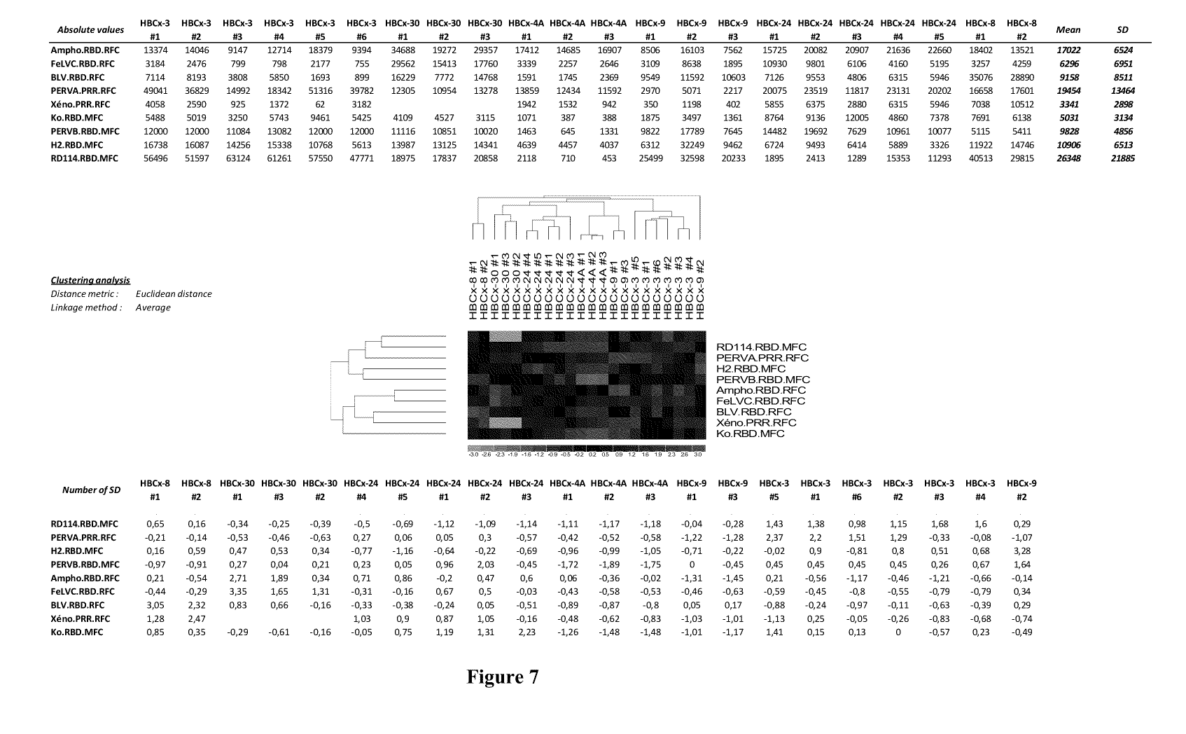

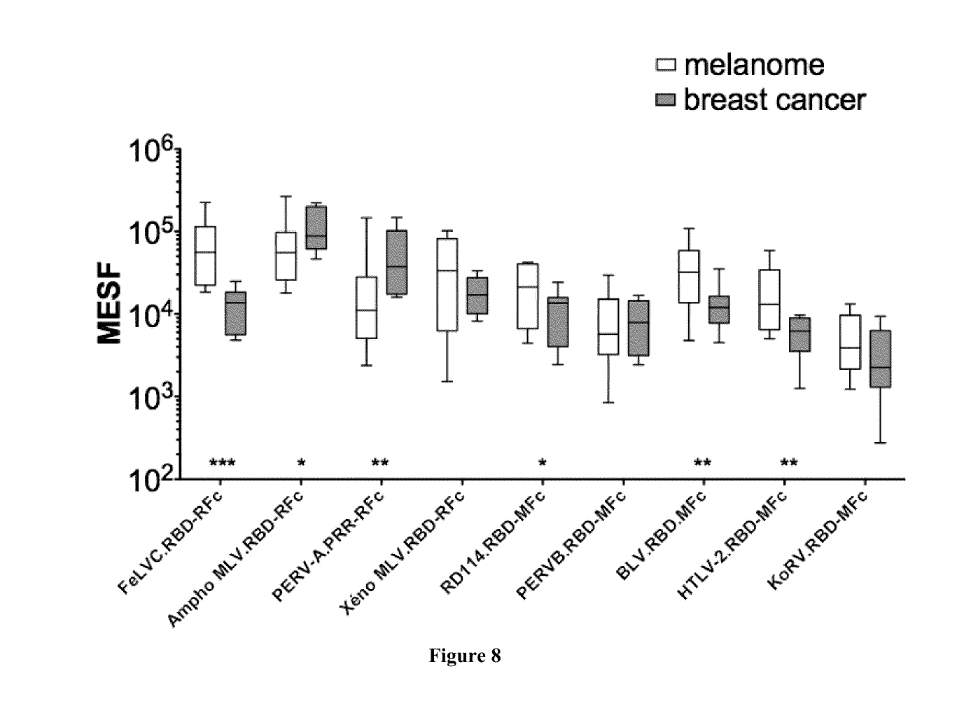

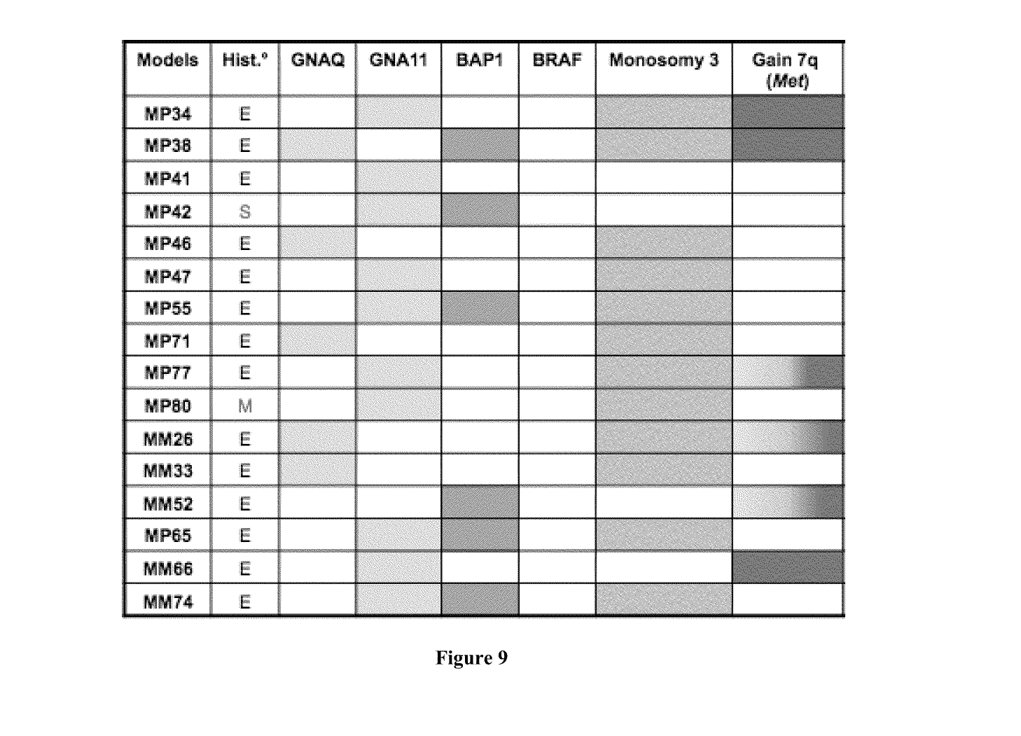

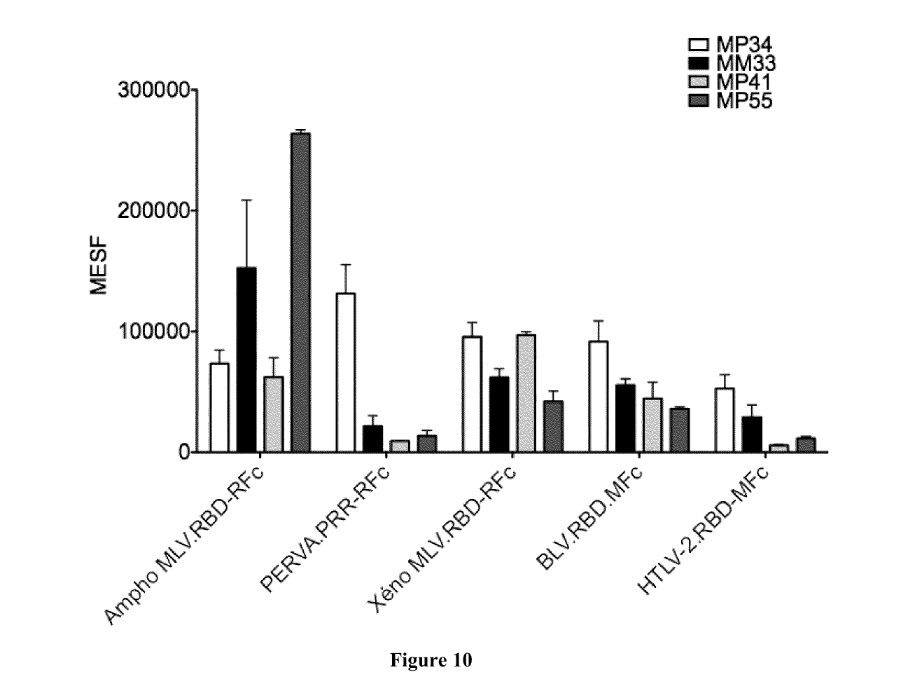

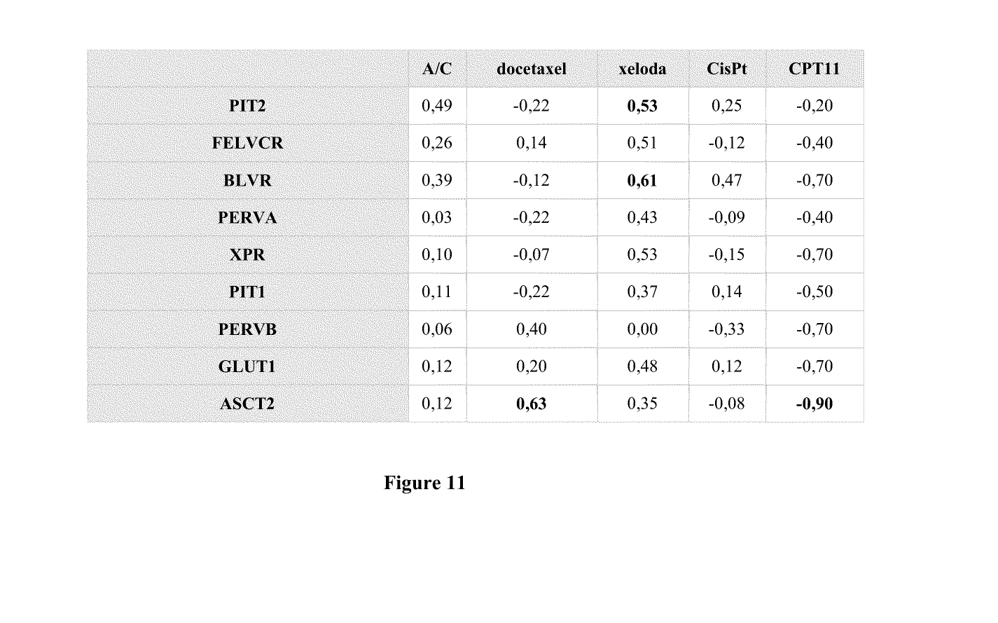

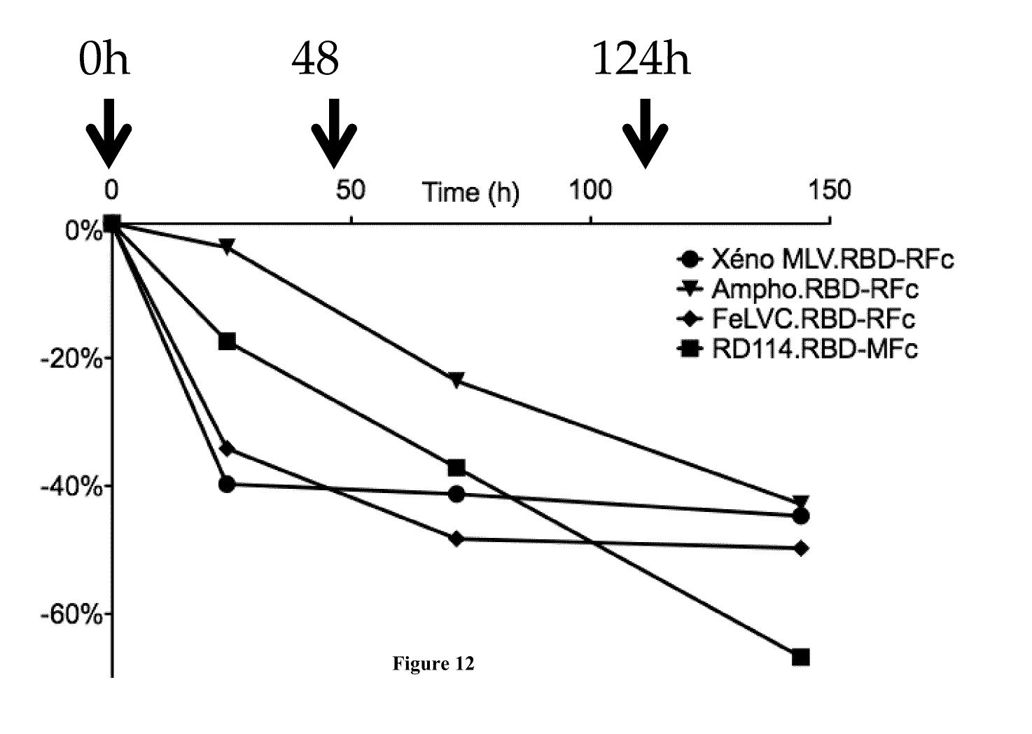

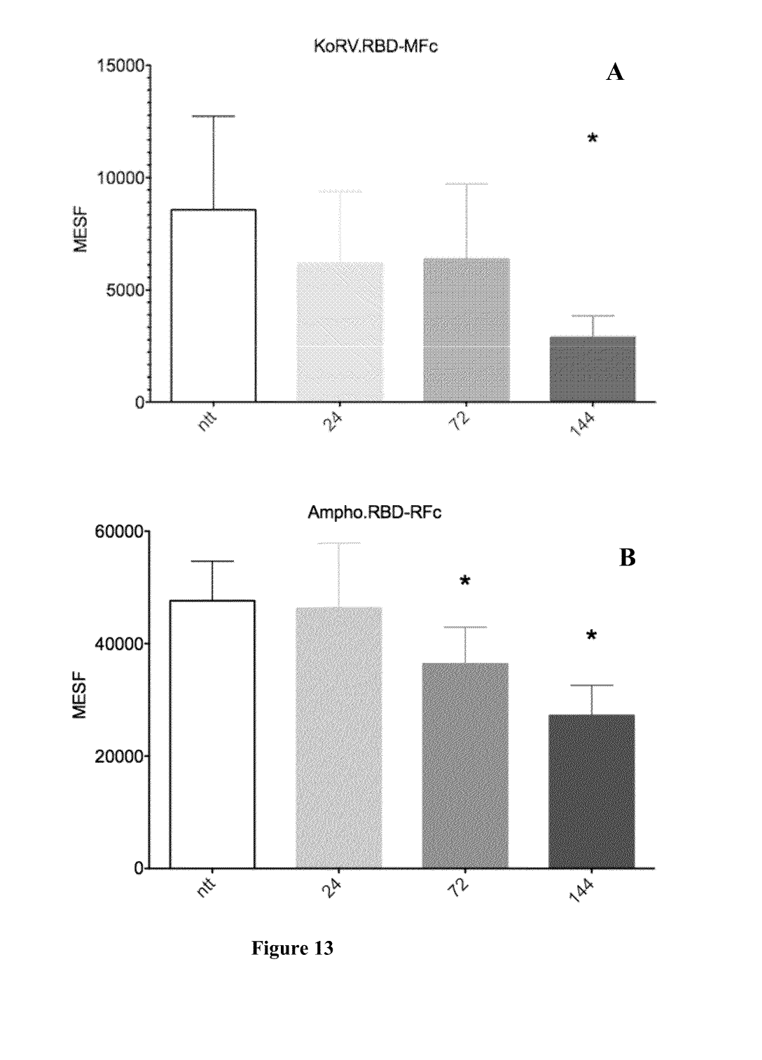

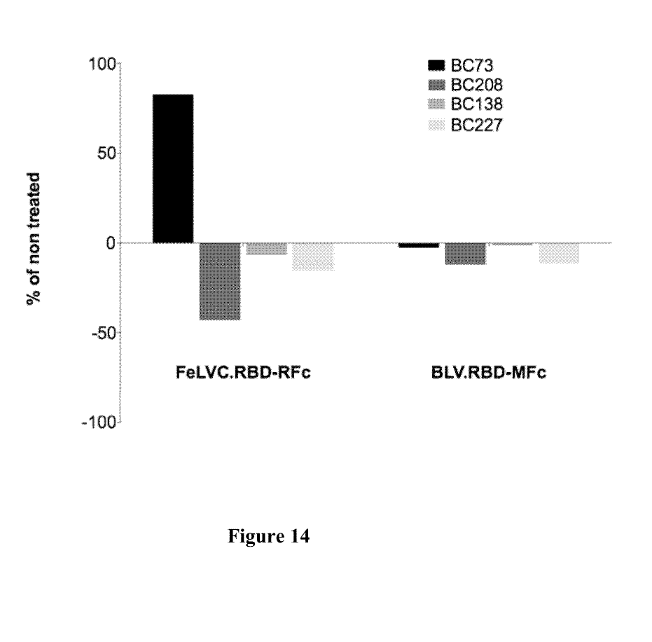

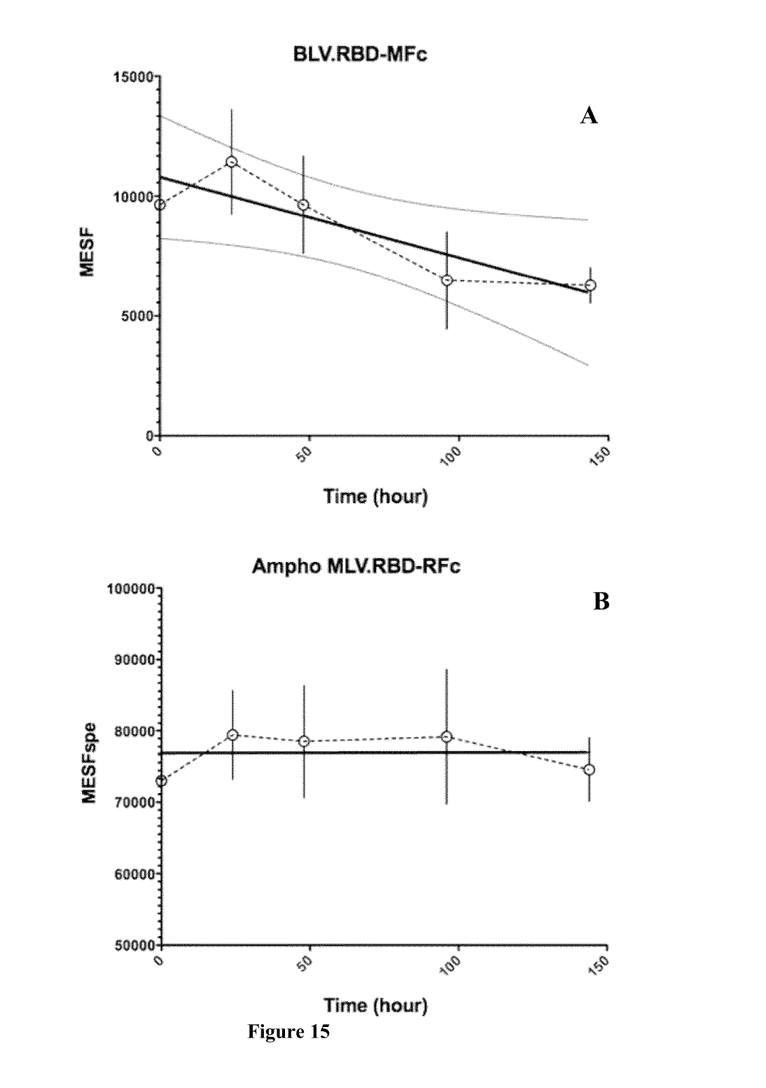

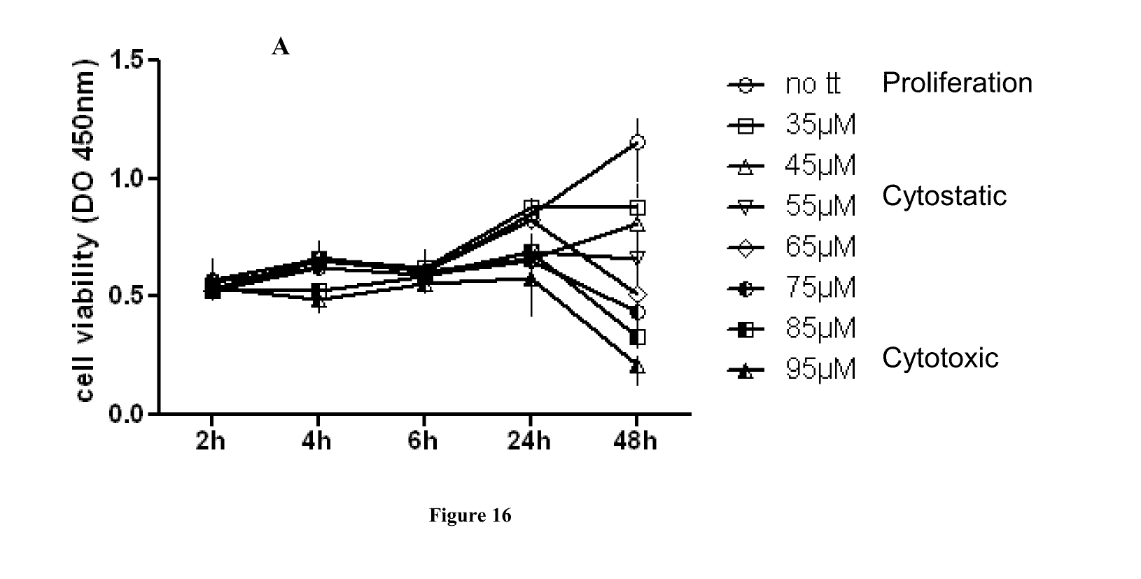

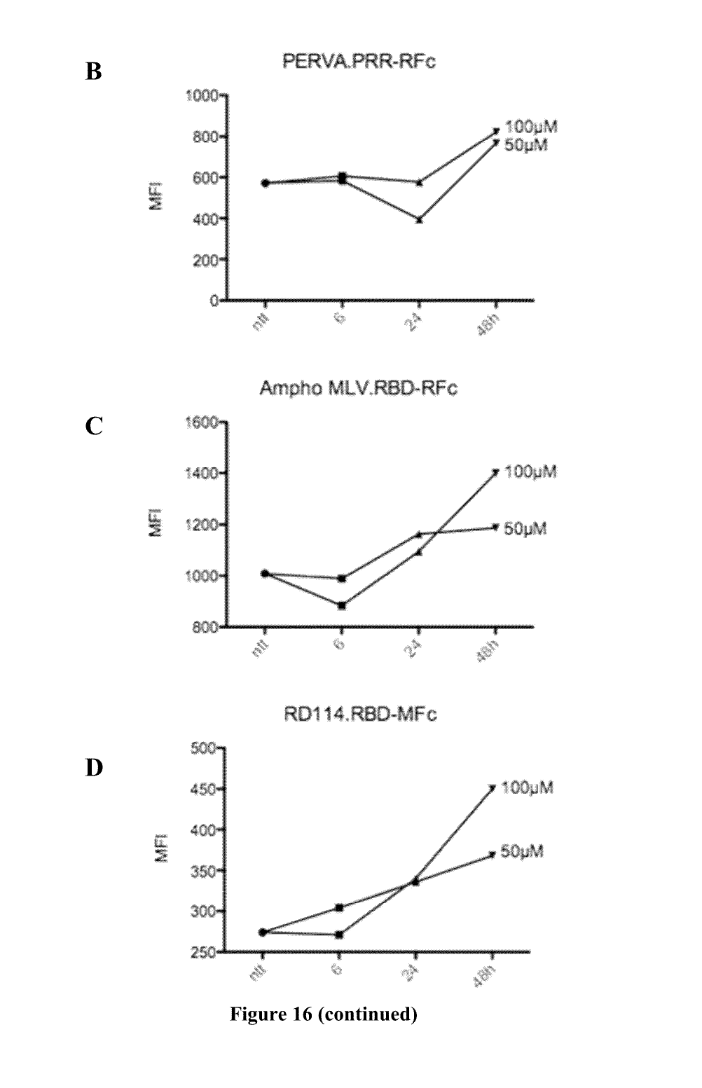

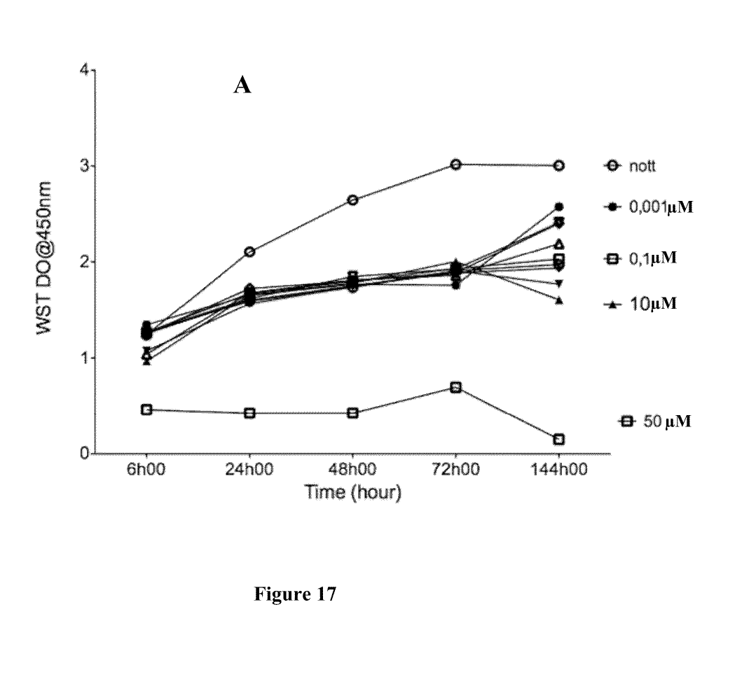

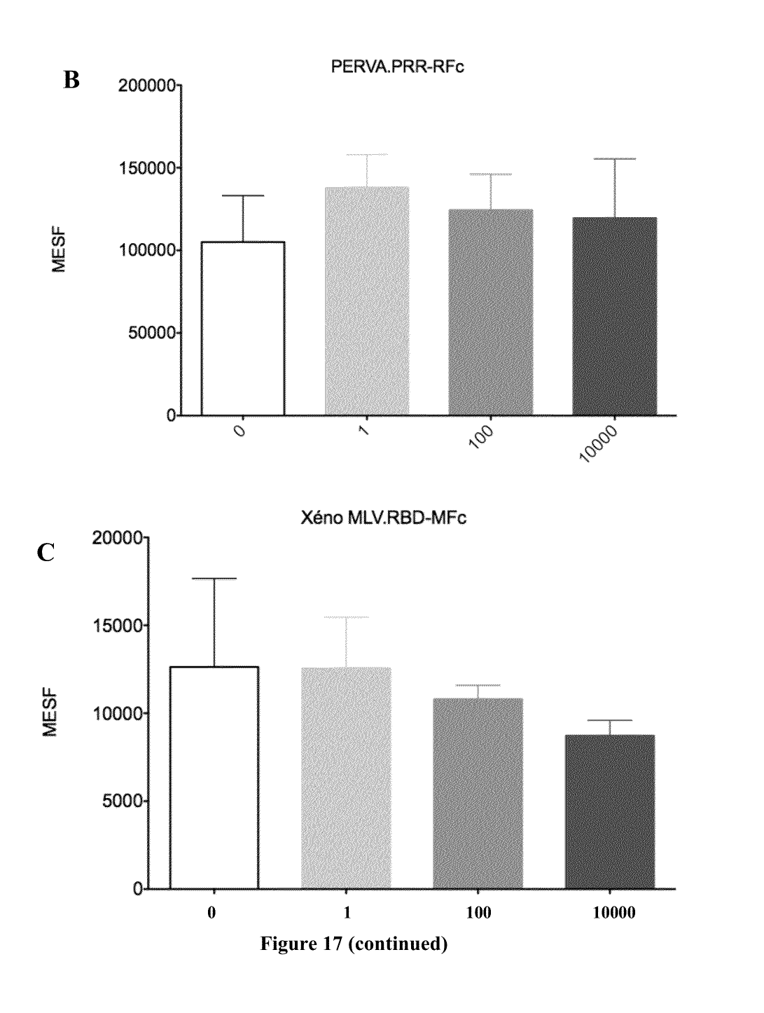

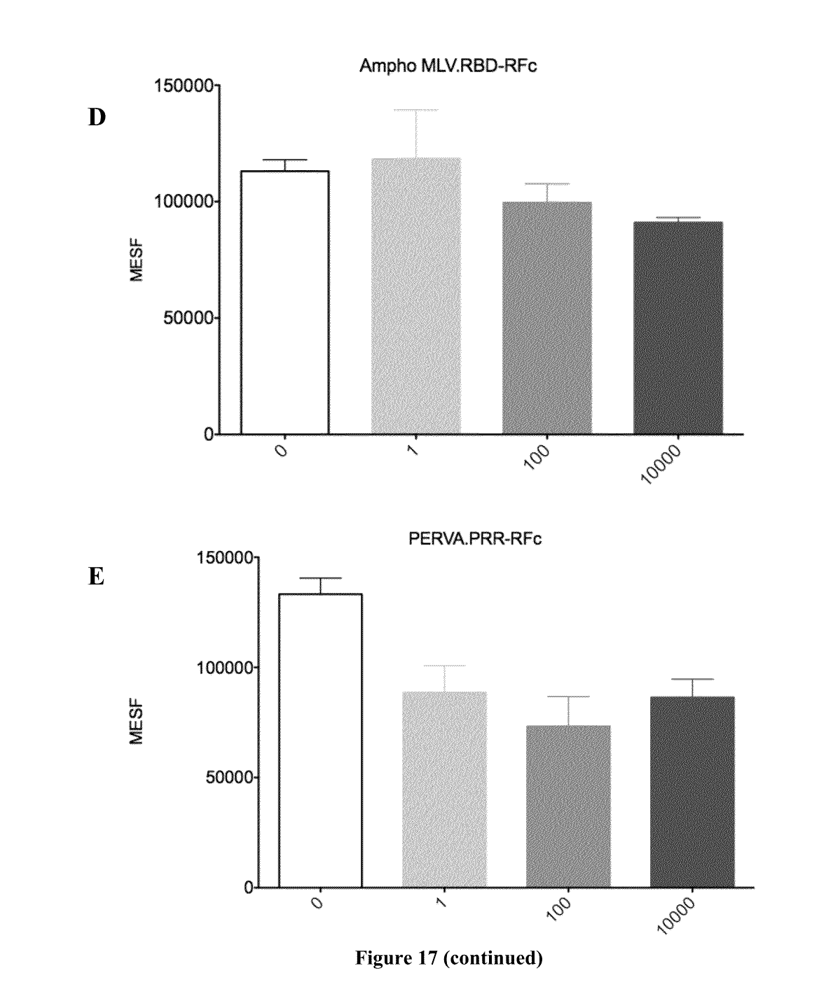

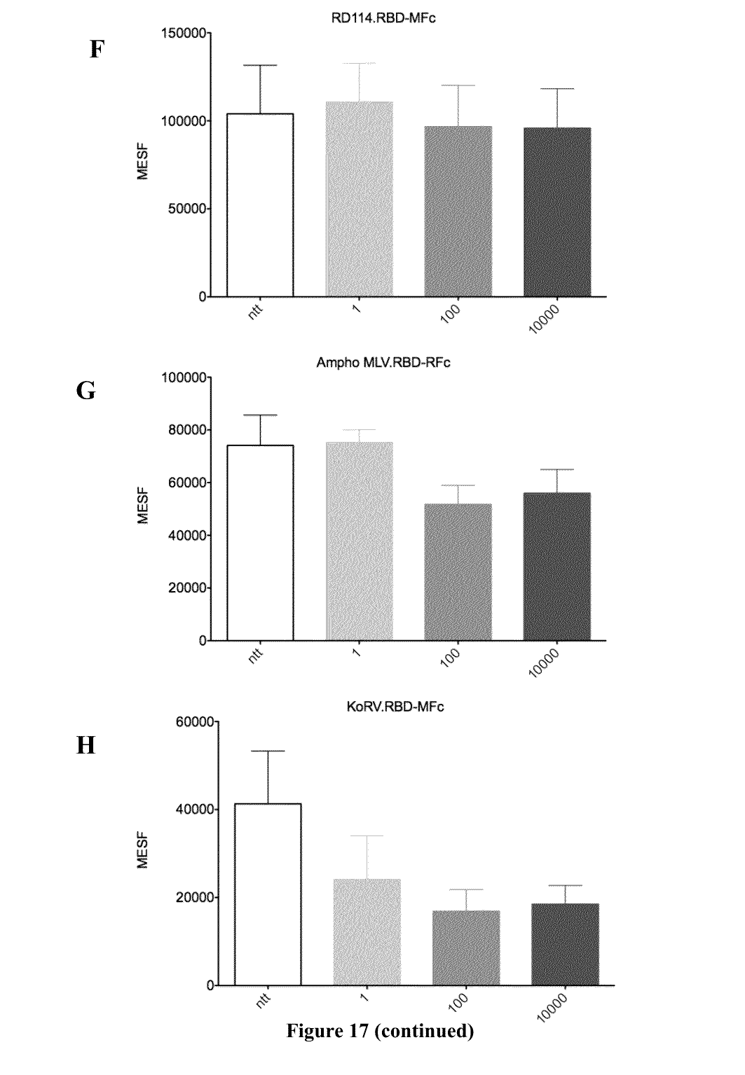

Each number of SD for a defined HBCx is represented by a square for each receptor binding ligand and presented at the center of the FIG. 7.

The scale under the representation is from left to right: −3.0, −2.6, −2.3, −1.9, −1.6, −1.2, −0.9, −0.5, −0.2, 0.2, 0.5, 0.9, 1.2, 1.6, 1.9, 2.3, 2.6, 3.0.

FIG. 8 presents the comparative detection of melanoma and breast cancer (BC) cells using various receptor binding ligands (Uvmel vs BC using a metric non paired t test: Mann Whitney U test).

x-axis: from left to right: FeLVC (SEQ ID NO:35).RBD-Rfc, AmphoMLV (SEQ ID NO:32).RBD-Rfc, PERV-A (SEQ ID NO:37).RBD-Rfc, Xeno (SEQ ID NO:34).RBD-Rfc, RD114 (SEQ ID NO:33).RBD-Mfc, PERVB (SEQ ID NO:38).RBD-Mfc, BLV (SEQ ID NO:41).RBD-Mfc, HTLV-2 (SEQ ID NO:40).RBD-Mfc and KoRV (SEQ ID NO:36).RBD-Mfc.

y-axis: MESF (molecule of equivalent soluble fluorophore)

In white: melanoma (corresponding to all the melanoma models used in the specification)

In grey: breast cancer (corresponding to all the breast cancer models used in the specification)

RBDs such as FeLVC, AmphoMLV, PERV-A, RD114, BLV and HTLV-2 allow to differentiate two types of solid tumor such as UvMel and breast cancer by their differential expression in two sets of solid tumors indicating a specific signature of cellular and/or tumor type.

FIG. 9 presents the characteristics of choroid melanomas (UvMel). (F. Nemati et al., Clin Cancer Res; 16(8), Apr. 15, 2010)
Hist: histology
   E: epitheloid type
   S: spindle type

GNAQ (heterotrimeric G protein alpha subunit) and GNA11 (Guanine nucleotide-binding protein subunit alpha-11) are proteins coupled to membranes receptors that activate various afferent ways and the mutations of which are exclusive and highly represented in choroid melanoma.

BAP1 is a tyrosine kinase that can be mutated with the concomitant loss of heterogeneity of chromosome 3(LOH).

BRAF: gene coding for B-Raf.

These characteristics are linked to the tumor aggressiveness.

FIG. 10 presents the specific signature of UvMel melanomas.
y-axis: MESF (molecule of equivalent soluble fluorophore)
x-axis: RBDs from left to right: AmphoMLV, PERV-A, Xeno MLV, BLV and HTLV-2

For each RBDs: histograms from left to right: MP34, MM33, MP41 and MP55

FIG. 11 presents a correlation matrix showing the predictive side of the response to a treatment by some RBDs.
A/C: Adriamycin and cyclophosphamide,
Docetaxel
Xeloda: capecitabine
CisPt: cisplatine,
CPT11: camphotecin.

Expression of RBDs in a set of breast tumor is correlated or not with the response of said set to anticancer drugs. Spearman rank correlation

Marked correlations are significant at p<0.05

Taking ASCT2 as an example, the more a RBD is expressed by a cell, the more it is sensitive to docetaxel but the less it is sensitive to CPT11.

FIG. 12 presents the effect of an everolimus treatment (2.5 mg/kg, i.p.—intraperotoneal) in a model of breast tumor xenograft (HBCx-3) known to be responsive to everolimus treatment at short times.

Mice of example 1 have been used.

Four RBDs are studied:
Circle: Xeno.RBD
Triangle: Ampho.RBD
Diamond: FeLVC.RBD
Square: RD114.RBD

The expression of RBDs varies significantly and more or less early with the treatment.

For example, Xeno and FeLVC vary at the beginning of the treatment while Ampho.RBD vary later and the variation of RD114 is higher than the one of Ampho.

FIGS. 13A to 13B present the results obtained with the treatment of FIG. 12 on various RBD (one treatment of everolimus 2.5 mg/kg, i.p. at 24 h, a second similar treatment at 24 h and a third similar treatment at 144 h.

FIG. 13A: KoRV.RBD.MFC

FIG. 13B: Ampho.RBD.RFC

FIG. 14 presents the effect of a cisplatin treatment (6 mg/kg, i.p.) in four models (one non-responsive, three responsive) of breast tumor xenograft.

The four models are the followings: HBCx-4A (non-responsive), HBCx-16 (responsive), HBCx-8 (responsive), HBCx-17 (responsive).

The effect is observed at 72 h post treatment with cisplatin.

Left histograms (FeLVC.RBD): from left to right HBCx-4A, HBCx-16, HBCx-8, HBCx-17.

Right histograms (BLV.RBD): from left to right HBCx-4A, HBCx-16, HBCx-8, HBCx-17.

For FeLVC, there is an increase of the expression of the RBD for the non responsive model.

There is a decrease of the expression of the RBD for the responsive model HBCx-16.

For BLV, there is no difference in the expression of the RBD for the responsive and non responsive models. This RBD is therefore not affected by cisplatin in these xenograft models.

FIGS. 15A and 15B present the effect of a radiotherapy treatment (4Gy) in a model (in vivo mice) of breast tumor xenograft HBCx-17.

The 4Gy dose has been used to obtain a moderate response of the tumor. At a higher dose, a too high decrease of the tumor is observed and the tumor becomes difficult to study by cytometry.

The effect has been observed at 24 h, 48 h, 72 h and 144 h.

FIG. 15A: BLV.RBD
x-axis; time (h)
y-axis: MESF

FIG. 15B: AmphoMLV.RBD
x-axis; time (h)
y-axis: MESF

Only BLV.RBD is responsive to the radiotherapy.

FIG. 16A to 16D presents the in vitro effect of cisplatin on a line coming from HBCx-4A and put on plastic in function of time.

FIG. 16A: cisplatine dose response kinetic

from top to bottom: white circle: no treatment (no tt), white square: 35 μM, white triangle (top point): 45 μM, white triangle (bottom point): 55 μM, white diamond: 65 μM, black circle:

75 μM, black square: 85 μM, black triangle: 95 μM.

When there is no treatment, there is proliferation of the line.

At 55 μM, the dose of cisplatin is a cytostatic dose.

At 95 μM, the dose of cisplatin is a cytotoxic dose.
x-axis: time in hours
y-axis: Cell viability (OD 450 nm)

FIG. 16B to 16D: three RBD studied with two doses of cisplatin (50 and 100 μM)

FIG. 16B: PERV-A.RBD
x-axis: time in hours
y-axis: MFI (mean of fluorescent intensity)

The expression of PERV-A is increased at 48 h at both doses.

FIG. 16C: AmphoMLV.RBD
x-axis: time in hours
y-axis: MFI (mean of fluorescent intensity)
The expression of MLV.RBD is increased at 24 and 48 h at both doses. At 100 μM, the RBD expression is higher than at 50 μM.

FIG. 16D: RD114.RBD
x-axis: time in hours
y-axis: MFI (mean of fluorescent intensity)

The expression of RD114.RBD is increased at 24 and 48 h at both doses. At 100 μM, the RBD expression is higher than at 50 μM.

FIG. 17A to 17H presents the in vitro effect of everolimus on a line coming from HBCx-4A and put on plastic in function of time.

FIG. 17A: everolimus dose response kinetic

from top to bottom: no treatment (nott), black circle: 0.001 μM, white square: 0.1 μM, black triangle: 10 μM, bottom white square: 50 μM.

At 0.001 μM, there is proliferation of the line.

At 0.1 μM, the dose of everolimus is a cytostatic dose.

At 10 μM, the dose of everolimus is a cytotoxic dose.
x-axis: time in hours
y-axis: WST (OD 450 nm)

FIGS. 17B and 17C: 6 hours after everolimus.

FIG. 17B: PERVA.RBD
x-axis; dose of everolimus (0, 1, 100, 1000 nM)
y-axis: MESF

FIG. 17C: XenoMLV.RBD
x-axis; dose of everolimus (0, 1, 100, 1000 nM)
y-axis: MESF

FIGS. 17D and 17E: 24 hours after everolimus.

FIG. 17D: AmphoMLV.RBD
x-axis; dose of everolimus (0, 1, 100, 1000 nM)
y-axis: MESF

FIG. 17E: PERV-A.RBD
x-axis; dose of everolimus (0, 1, 100, 1000 nM)
y-axis: MESF

FIGS. 17F to 17H: 72 hours after everolimus.

FIG. 17F: RD114.RBD
x-axis; dose of everolimus (0, 1, 100, 1000 nM)
y-axis: MESF

FIG. 17G: AmphoMLV.RBD
x-axis; dose of everolimus (0, 1, 100, 1000 nM)
y-axis: MESF

FIG. 17H: KoRV.RBD
x-axis; dose of everolimus (0, 1, 100, 1000 nM)
y-axis: MESF

The response of RBDs to everolimus is time and dose dependant.

### EXAMPLES

### Example 1

### Engrafted Human Tumors in Mice

The human breast cancer specimens were obtained with informed consent from the patients undergoing surgery. Fresh tumor fragments were grafted into the interscapular fat pad of 8-12 week old female Swiss nude mice, under avertin anaesthesia. Mice were maintained in specific pathogen-free animal housing (Institut Curie, Paris, France) and received estrogen (17 mg/ml) diluted in drinking water. Xenografts appeared at the graft site 2 to 8 months after initial transplantation. They were subsequently transplanted from mouse to mouse and formally established since third in vivo passage (Marangoni, Elisabetta A new model of patient tumor-derived breast cancer xenografts for preclinical assays Clin Cancer Res. 2007 Jul. 1; 13(13):3989-98). All the experiments were performed in accordance with the UKCCCR guidelines about animal ethics in neoplasia research (British Journal of Cancer (2010) 102, 1555-1577). Present experiments were all performed in well-established tumor models and required a minimal tumor size of 600 mm³ to collect enough cellular material. In all cases, particular attention through careful clinical examination has been paid to ensure that the recipient mice remained in a general good health to avoid as far as possible necrotic tumors. All tumors were aseptically excised from ethically sacrificed mice with autoclaved surgical material. NEDB and culture media w/o FCS are both equivalent for a short period before processing.

### Example 2

### Primary Cells Isolation

Tumors were obtained immediately after excision from mice and conserved in cold culture media to avoid desiccation and cell death until processing. Specimens were trimmed to remove surrounding breast and fat tissue, cut into 2-4 mm small pieces with a scalpel, crushed in a non-enzymatic dissociation buffer and incubated at 37° C. for 30 min. Resuspended tumor pieces were aspirated up and down with a 1000 μl micropipette mounted with a cut-end tip every 10 min, so that tip's diameter was adapted to tissue fragments size along the dissociation. Samples were sieved through a 40 μm nylon mesh (cell strainer BD Bioscience). Recovered cells were centrifugated and resuspended in dissociation medium consisting in CO₂ independent media (Gibco) complemented with 30% of heat inactivated fetal calf serum and stored at 4° C. until next step. This first non-enzymatic dissociation step was repeated with remaining tissue fragments. For enzymatic dissociation step, mixture was incubated for 30 min at 37° C. with collagenase III (Sigma Aldrich) and deoxyribonuclease I (Sigma Aldrich) both at 200 U/mL in dissociation medium, and then 40 μm sieved. Once again, recovered cells were resuspended in dissociation medium and stored at 4° C.

All cell fractions were pooled, carefully layered onto the upper of a double gradient Ficoll™ (Histopaque from Sigma-Aldrich d=1,077 and d=1,119) as described by the fabricant for leukocyte separation (procedure NO 1119) and spin at 700 G for 30 min at room temperature. Ring layers were washed twice in CO₂ independent medium and can then be stored at 4° C. before use (storage up to overnight will not alter significantly the cell viability). Except for the Ficoll™ step, spinning was set at moderate speed for short time (1200 RPM-2 min).

### Example 3

### Cell Viability and Culture

Trypan blue exclusion count was performed in Malassez slide to estimate cell viability and calculate the total and viable cell yields immediately after ending up the dissociation protocol.

For ex vivo viability culture assay, 40 000 cells were plated in flat bottom 96 well plates in 100 μl of DMEM High glucose concentration supplemented with glutamine, sodium pyruvate, penicillin and streptomycin, and 10% of fetal calf serum and incubated at 37° C. 10 μl of WST-1 (based on the cleavage of a tetrazolium salt, Roche diagnostic) was added to six wells at different time points to monitor cell viability over time. Drawing optical density at 450 nm against time gave a proliferation curve reflecting viability of isolated primary cells. At end point, cells were harvested by trypsinization and stained for mouse versus human marker to check if viability was due to human tumor or mouse stromal cells.

In vitro treatments with anti-cancer drugs were performed in flat bottomed 96 well microplates. Equal number of viable cells (40 000) were distributed in each well and allowed for attachment for 3 days. Medium was renewed and drugs (Docetaxel, Avantis Pharma; Cisplatin, Merck) added at incremental concentrations. After 72 h incubation in presence or absence of compounds, WST-1 (based on the cleavage of the tetrazolium salt by respiratory mitochondrial enzyme, Roche diagnostic) was added and incubated following manufacturer instructions. Optic density was read on a microplate reader at 450 nm. A sigmoid analytical curve gave the 50% inhibitory concentration (IC50).

### Example 4

### Receptor Binding Ligands Generation and Production

H2.RBD.mFC, Ko.RBD.mFC, RD114.RBDmFC and A.RBD.rFC immunoadhesins were derived from HTLV-2, KoRV, RD114 and A-MLV SU, respectively: (SEQ ID NO:40), (SEQ ID NO:36), (SEQ ID NO: 33) and (SEQ ID NO: 32) and fused to either mouse or rabbit IgG Fc fragments. All constructs were inserted into the eucaryotic expression pCSI vector (Battini et al, 1999). 293T cells grown on poly-D-Lysine coated surface were transfected by calcium-phosphate precipitation method, washed 16 hour later and incubated for another 48 hours in serum free Optipro SFM (Invitrogen) supplemented with glutamine and non essential aminoacids. Conditioned media were harvested, filtered through 0.45 μm pore-diameter filters and concentrated 100-fold by centrifugation at 3600 rpm per minute on 9 kDa cut-off Icon concentrators (Pierce). Samples were aliquoted and stored at −80° C. Each preparation was verified for integrity by immunoblotting and immunoadhesin concentration was measured by ELISA using anti rabbit or anti mouse IgG Fc.

### Example 5

### Immunostainings for Flow Cytometry

For flow cytometric analyses, immunostainings were performed as follow: 3.10⁵ to 5.10⁵ viable cells were stained in binding buffer (consisting in PBS with 2% HIFCS). All antibodies were used in one step as all were primary conjugated. Rat anti mouse Pan H-2 (CMH-I) PE conjugated (Biolegend) and mouse anti-human EpCAM (CD326) PerCpCy5.5 conjugated (Biolegend) were used to discriminate between mouse and human cells. Mouse anti-human CD44 APCH7 conjugated (BD bioscience) was used to define proportion of CD44-positive cells among human cell population. After 20 minute incubation at 4° C. protected from light followed by two washes, 4′,6′-diamidino-2-phenylindole (DAPI from Invitrogen) was added to each sample (at 2 ng/mL) just before FACS reading to exclude dead cells. In some cases, cells were also fixed after staining in PFA 0.05% overnight until data acquisition. All antibodies were separately titrated with dissociated cells from BC xenografted models.

For metabolite transporters labelling, 200 000 to 500 000 viable cells were incubated at 37° C. in RBD premix consisting in 3 μl of a RBD mouse Fc tagged plus 3 μl of a RBD rabbit Fc tagged diluted in 50 μl of CO₂ independent medium and washed once before secondary staining with fluorescent conjugated antibodies against mouse (goat anti-mouse AF488, Cell Signalling Technology) and rabbit (goat anti-rabbit AF647, Cell Signalling Technology). Rat anti-mouse Pan H-2 added in a third step allowed for the discrimination between human and mouse cells.

### Example 6

### Flow Cytometry Data Acquisition and Processing

To compensate overlapping fluorescence, we used compensation beads comprising positive and negative populations (Invitrogen anti-Mouse and anti-Rat/Hamster depending on antibody type). In view to set-up PMT and in-silico auto-compensated acquisition and given highly variable cell auto-fluorescence in the wide optical spectrum, isolated primary cells were used as universal unstained control.

In order to set a threshold for positive events involving non-specific binding due to isotype or fluorescent dye and to avoid artefactual shifting of stained population in dot plot representation, each antibody was confronted to its own isotype in whole fluorescent context. In these tubes all specific antibodies were present as in the full panel tube except for the one of interest thus replaced by its isotype (“Fluorescence minus one”) (Roederer M. Compensation in flow cytometry. Curr Protoc Cytom. 2002 December; Chapter 1: Unit 1.14).

High sensitivity is a challenge in flow cytometry. A way to reach this goal is to pay a particular attention to standardization of each point of experiment from dissociation to staining protocols. To visualize thin variation of expression level in multiple channel protocol, we need one unique unit, as fluorescence arbitrary units read on the same cytometer from two different fluorophores in their own channel cannot be confronted. Molecule of Equivalent Soluble Fluorochrome (MESF) is given by regression of arbitrary fluorescence measured versus standardisation beads coated with known amounts of same fluorochrome used in cells staining (AF647 and AF488 MESF quantum beads, BangsLab) acting as intra- and interexperiments calibrator. Measured fluorescence depends on lasers power, photomultiplicator tube settings, pH of staining medium. Fluorescence measured is proportional to the number of fluorochromes linked to the antibody used, itself correlated to the number of antibody bound to specific sites. With MESF we abolish most of named bias resulting in variation of measure due to floating brightness of fluorescent molecules. For RBDs, quantification of non-specific signal was determined using secondary conjugated antibody alone.

Fow cytometry fcs3 data files were collected on a LSRII (Becton Dickinson) flow cytometer by acquiring enough events number to visualise small/side populations. These data files were exported in Macintosh version of Flowjo 9.1 to refine gating to single live cell population and further analysis. Geometric mean of fluorescence was chosen for markers descriptive statistics.

### Example 7

### Statistical Analysis

The nonparametric Kruskal-Wallis test was used for inter-group comparison for each transporter expression level.

### Example 8

### Immunohistochemistry

Samples were fixed in formalin (10% vol/vol in PBS), ethanol dehydrated, and paraffin embedded. Sections of 4-μm width were prepared and stained with hematoxylin eosin safranin (HES) according to standard histological procedures.

To perfom immunostaining, the 4-μm tumor sections were adhered to Superfrost Plus slides (Fisher Scientific, Pittsburgh, Pa.) and heated at 40° C. at least 4 hours. Sections were deparaffinised overnight, rehydrated through graded alcohols until distilled water. Unmasking was done in citrate buffer 10 mM, pH 6, by heating 20 min with 350 W microwave. Immunostaining was performed in NEXES automate from VENTANA Medical Systems (ROCHE Diagnostics). Primary rabbit antibody against CD44 (Sigma HPA005785), diluted 1:200, was incubated on slides 32 min at 37° C. Avidine-Biotine system and peroxydase revelation was used with DAB as substrate. Slides were counterstained with hematoxylin. Images were captured using a fully motorized microscope (Zeiss Axio Imager Z1) with high resolution color digital camera (Zeiss HRc) and AxioVision 4.6.3 SP1 software.

### Example 9

### Tissue Dissociation for High Recovery Yield of Viable Cells

In order to concomitantly increase total recovery yield and cell viability, different combinations of dissociation steps using enzymes cocktails, non enzymatic dissociation buffer (NEDB), or both, were tested, but always proceeded in no more than two to three consecutive incubations. As a general rule of thumb, incubation time was limited to 20 min at 37° C.; between each incubation step, isolated cells were recovered by total digested tissue sieving, the remaining tissue being incubated a next step further with fresh enzymes or NEDB cocktail. Each recovered fraction was washed and kept in culture medium supplemented with 30% FCS at 4° C. Fractions were then pooled before cell labelling or culture. This procedure avoid cells to be exposed too long to enzymes and to be maintained as far as possible at 4° C., altogether to maximise cell viability.

Four different protocols were hence compared on four different breast cancer xenografts. Basically, three steps were performed that can rely either on non-enzymatic dissociation using a chelator cocktail (NEDB), on classical enzymatic digestion, or on a combination of both. The first protocol consisted in three enzymatic steps (collagenase III, DNase I), the second in three enzymatic steps using the same cocktail supplemented with trypsin, the third in two dissociation steps using NEDB followed by a final enzymatic digestion incubation (collagenase III and DNase I), and the fourth was identical to the third except that trypsin was added to the other enzymes (Table I). As shown in the Table I, NEDB dissociation followed by a final enzymatic dissociation step was repeatedly the most efficient protocol regarding the yield of viable cells recovery as determined by trypan blue staining when normalized per gram of starting tissue; replacing enzymes by NEDB for the two first dissociation steps was followed by a two fold increase in absolute count of the cells of interest. With such a dissociation protocol, about 3.6×10⁷ viable cells could be obtained, depending on the xenografted model used. Adding trypsin to enzymatic cocktails did not significantly increase the total recovery yield in both protocols, but increased viability of recovered cells. This effect could be due to digestion of necrotic cells by trypsin, hence increasing representation of viable cells in the final samples.

The third dissociation protocol was next adopted, i.e. two NEDB dissociation steps followed by a final enzymatic dissociation without trypsin, as in the optimized protocol. It is noteworthy that we did not go through the complete dissociation with all processed models at the end of the different protocols. This decision was made to limit the dissociation time, based on the fact that subsequent enzymatic steps did not dissociate much more viable cells while concomitantly altering viability of recovered cells.

TABLE I
Optimization of the dissociation and purification steps for single live
cells recovery. Recovery yields and viability (trypan blue) obtained
through different tissue dissociation and cell purification protocols.
Viable cell number (×10⁶) per gram of tumor (% viability, trypan blue).

| | | | | | | | NEDB + | |
|---|---|---|---|---|---|---|---|---|
| | | | ENZYMES + | | NEDB + | | Enzymes + | |
| ×10⁶ cells | Enzymes | | Trypsine | | Enzymes | | Trypsine | |
| HBCx-1 | 38 | (51) | 39 | (47) | 75 | (58) | 72 | (56) |
| HBCx-4A | 2 | (10) | 7 | (14) | 11 | (10) | 9 | (13) |
| HBCx-14 | 11 | (22) | 6 | (14) | 13 | (24) | 11 | (32) |
| HBCx-15 | 10 | (51) | 19 | (65) | 44 | (46) | 28 | (61) |
| | Before | | High density | | Low density | | Dual density | |
| | FICOLL | | FICOLL | | FICOLL | | FICOLL | |
| HBCx-9C | 48 | (29) | 30 | (72) | 22 | (77) | 31 | (89) |
| HBCx-9F | 32 | (23) | 23 | (85) | 24 | (90) | 29 | (78) |
| HBCx-22 | 30 | (42) | 20 | (68) | 19 | (72) | 19 | (75) |
| HBCx-41 | 26 | (50) | 13 | (55) | 10 | (53) | 13 | (56) |

### Example 10

### Dual Density Gradient Ficoll™ Purification of Viable Cells

For red cells and debris elimination, we chose to perform a Ficoll™ extraction of viable cells and tested two different Ficoll™ densities, 1,077 and 1,119 referred as low and high density Ficoll™ respectively (Table I). Whereas high density Ficoll™ retained more viable cells, low density Ficoll™ appeared more efficient for viable cells purification as shown by the relative proportion of viable cells following extraction. However, since a tumor was intrinsically composed of heterogeneous cells based on their respective densities (aneuploid, diploid, polyploidy cells), all of them having to be recovered for subsequent analyses, we then tested a dual density Ficoll™ gradient (1,077 plus 1,119) that might concomitantly separate and purify aneuploid and polyyploid cells. Fractions were pooled and viable (trypan blue exclusion) cells counted. Total recovery yield of viable cells was slightly but repeatedly higher using the dual density Ficoll™ purification than either a low or a high density Ficoll™ alone (Table I). Debris and red cells elimination was slightly better when proceed with 1,119 or dual compared to low density.

### Example 11

### Reproducibility of the Finalized Protocol (Recovery Process of the Invention)

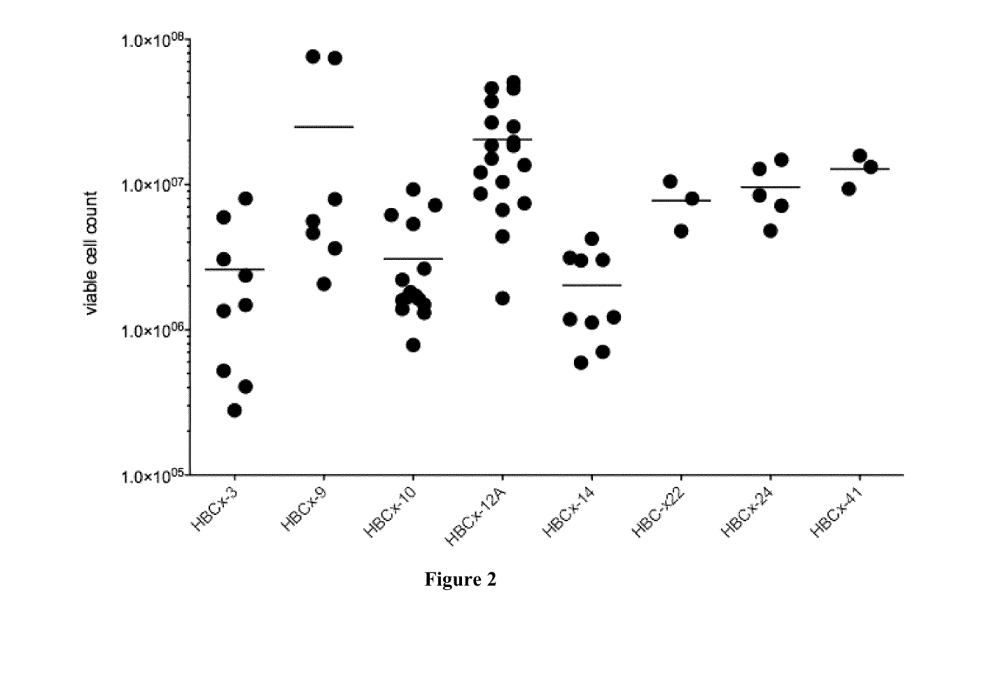

To validate the reproducibility of this finalized protocol, i.e. two NEDB dissociation steps followed by a final enzymatic dissociation and purification of cells through a dual density Ficoll™, it was applied on twelve different xenografted tumors of the same preclinical breast cancer model (HBCx-10), giving meanwhile 2.10⁶ viable cells per gram of tumor, along with a standard deviation of 68% (1.4.10⁶). When tested on 6 different tumor models, that altogether represent 28 dissociated xenografts, the mean recovery yield reached 1.38.10⁷ viable cells per gram of tumor. As shown in the FIG. 2, the total cell recovery was well reproducible among each model, but ranged from 8.10⁵ to 8.10⁷ viable cells per gram of tumor. These results therefore point out the high reproducibility of the finalized protocol for a given model, but also the large heterogeneity between xenografted models of a similar tumor type. Of note, these yields can be obtained for tumor size ranging from 600 to 2 500 mm³, 600 mm³ being a minimum threshold under which we repeatedly failed to obtain sufficient live cells for subsequent analysis.

### Example 12

### Multiparameter Staining and Flow Cytometry Analyses

The above detailed procedure allowed us to perform immunostaining for surface antigens and analyses by flow cytometry of recovered viable cancer cells. Indeed, for this peculiar application, the elimination of red blood cells, debris, and part of necrotic cells achieved through the purification step was of prime importance to perform antibody labelling for easier cytofluorometric analyses and/or cell sorting, and to limit apoptosis/necrosis factor concentration in the medium that might alter long term cell viability.

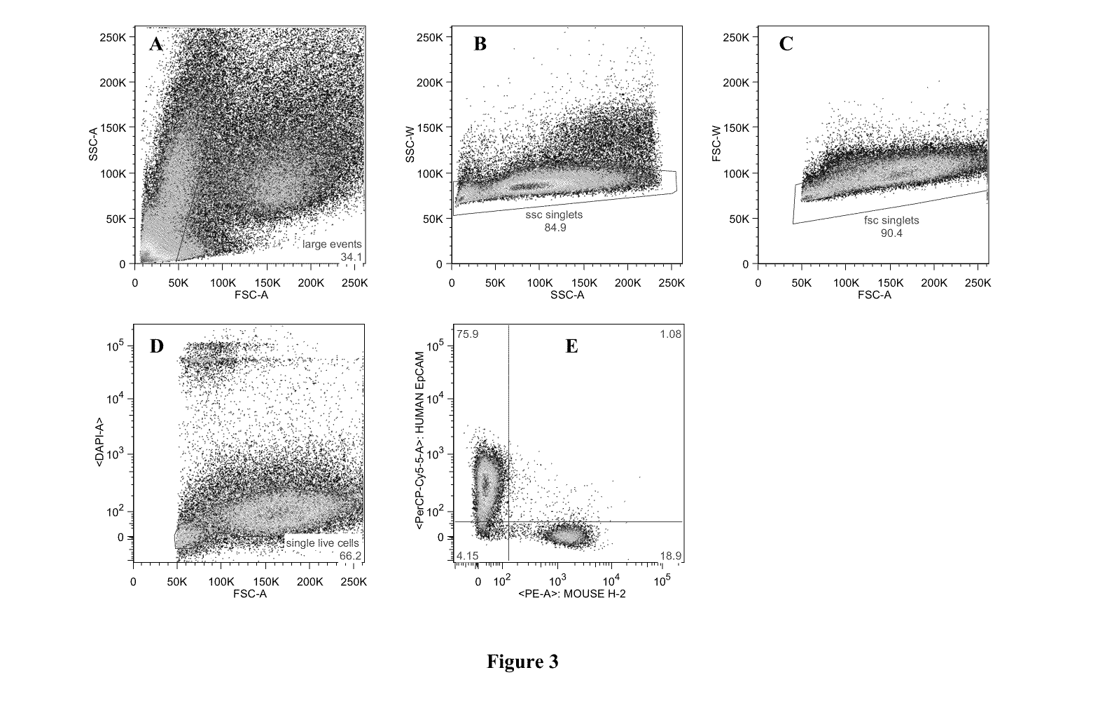

The gating strategy consisted in excluding cell debris, cell aggregates, and dead cells identified by positive DAPI nuclei staining (FIG. 3A to 3D). Thereafter, to discriminate human tumor cells to mouse stroma cells, an anti pan-H2 antibody directed against most murine MHCI molecules was used. Murine cells were readily identified and could therefore be either excluded from the analyses (FIG. 3E), or studied as tumor micro-environment.

### Example 13

### Viability of Studied Cells by Flow Cytometry

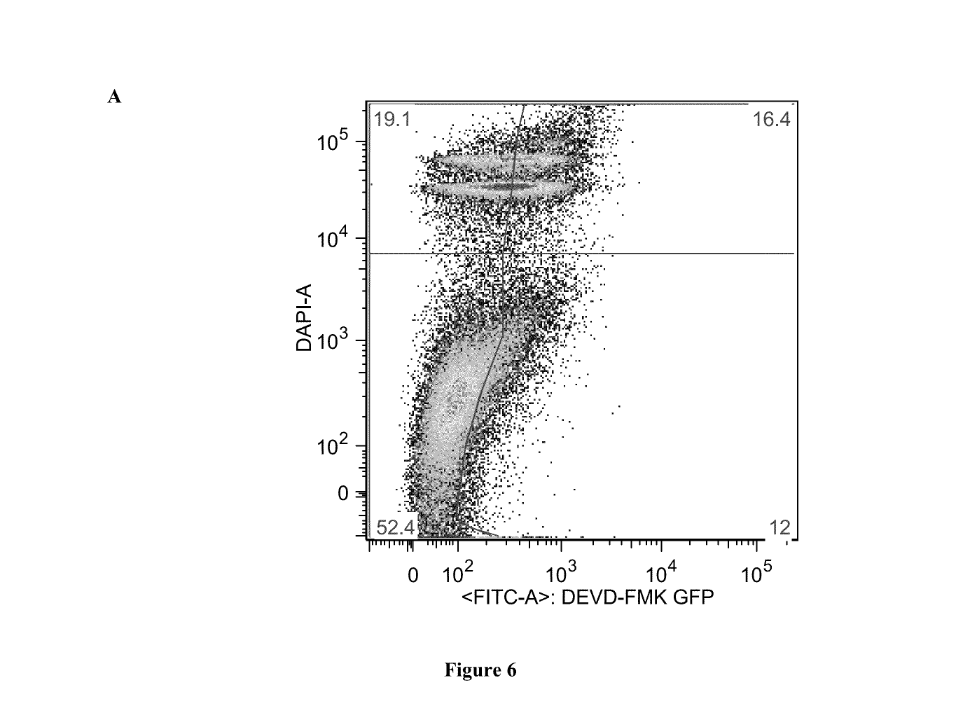

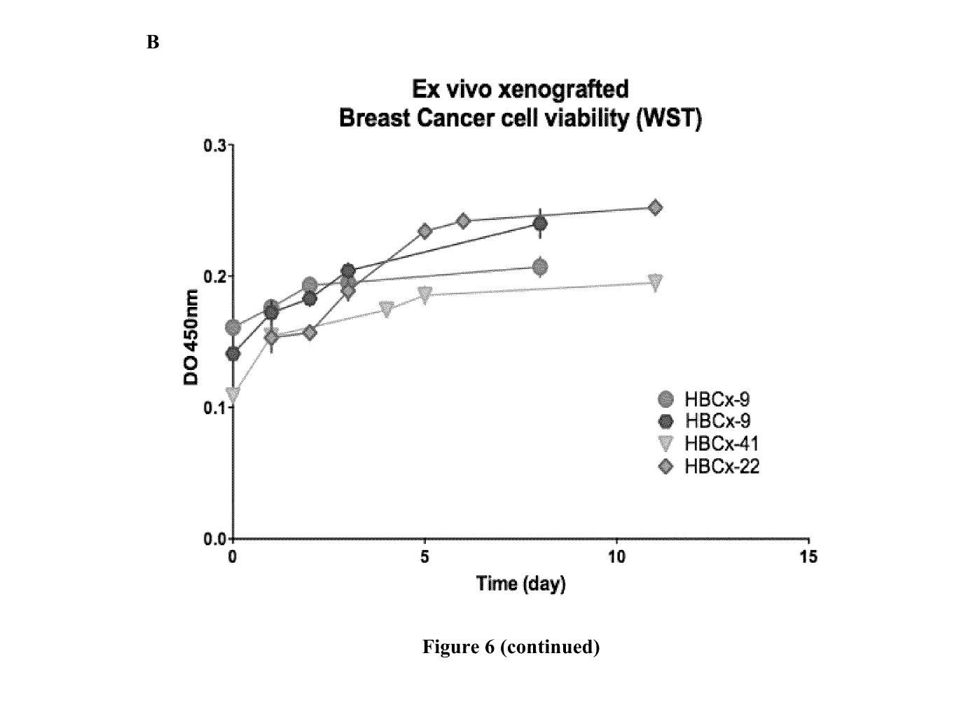

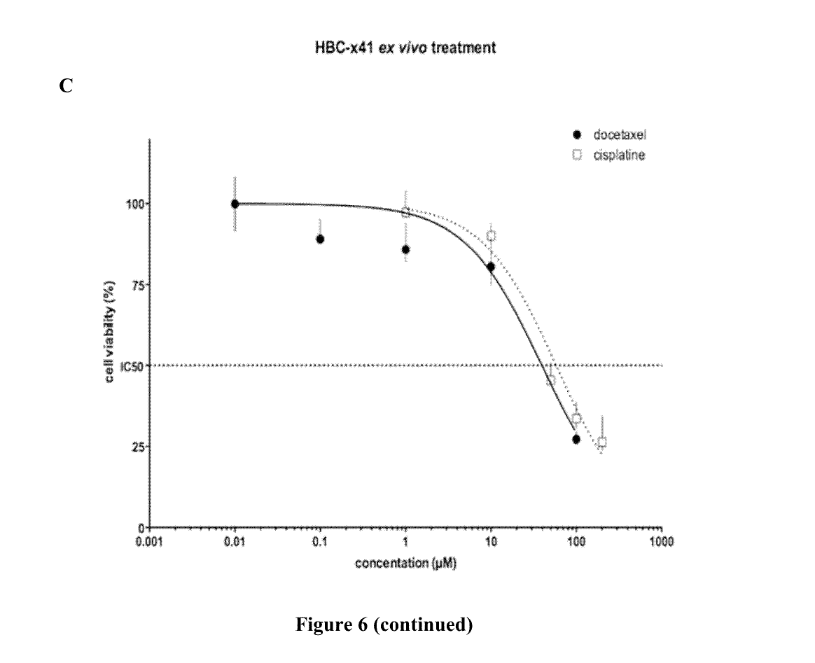

To validate our selection process of viable cells, we tested in which extent the dissociation protocol induced cell suffering by monitoring apoptosis through active caspase 3 staining. As shown in FIG. 6A, less than 10% of viable cells (DAPI negative) displayed an activated caspase 3.

As an ultimate read-out of cell viability, we finally cultured isolated cells recovered through the above protocol in DMEM under plastic-adherence conditions more than 12 days. Cells remained viable for a long time, as revealed by a WST assay (FIG. 6B); furthermore, ex vivo cultured cells were sensitive to common doses of anti cancer drugs in a dose response manner, reflecting physiological response to treatment comparable to commonly used established cell lines (FIG. 6C).

### Example 14

### Comparison of Flow Cytometry Versus Immunohistochemistry (IHC) Data

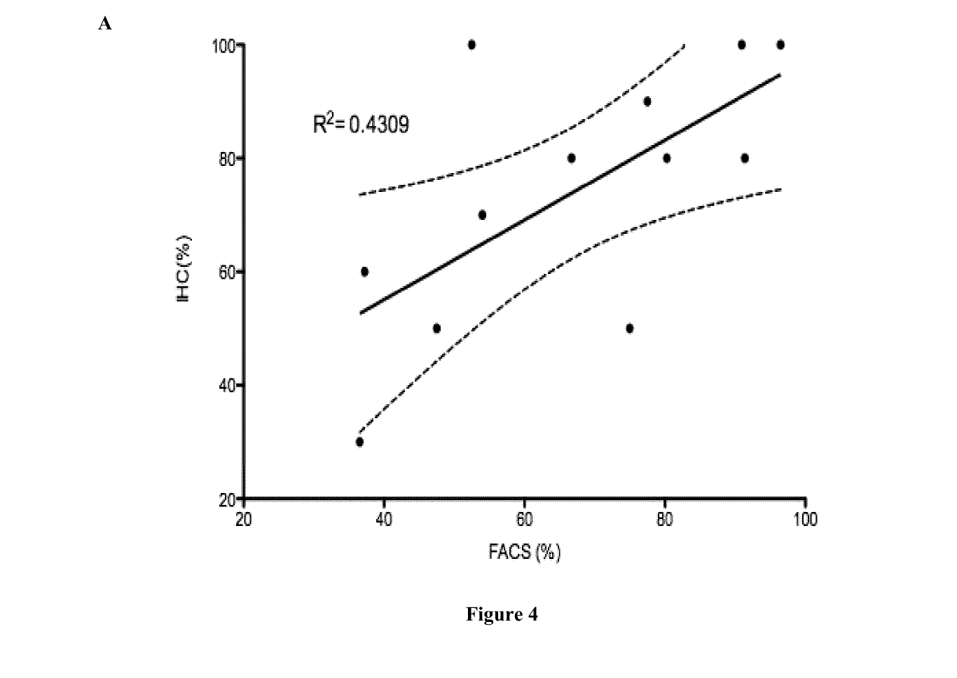

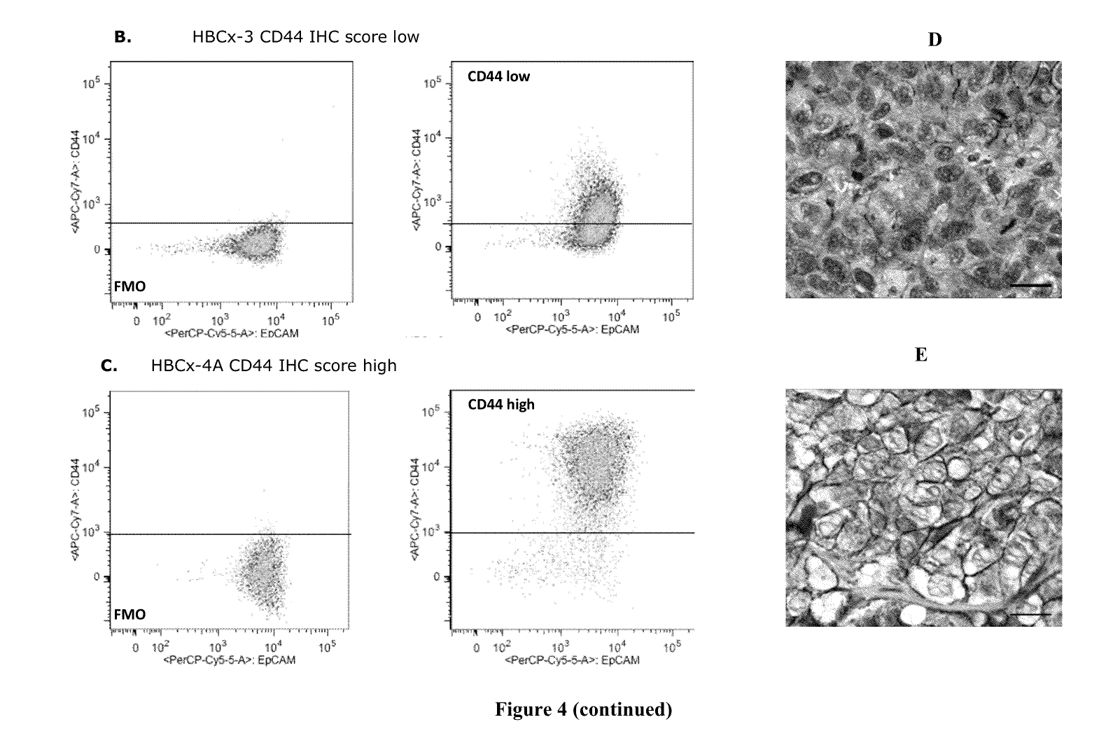

To validate flow cytometric data obtained after the finalized protocol (recovery process of the invention) and assess whether tissue dissociation could significantly modify cellular representation or markers expression, we then compared immunohistochemical and cytofluorometric labellings of human tumor cells using the cell surface expression of the CD44 marker. In this, cell surface status of CD44 was assessed by flow cytometry and compared to IHC on a panel of 12 different human breast cancer xenografts. The respective proportions of CD44 positive cells obtained by both techniques were compared and it was concluded that the proportion of CD44 positive cells were similar, as shown in FIG. 4A. Similarly, labelling intensities, as determined visually in IHC and through relative fluorescence intensity in flow cytometry, were comparable as illustrated in FIGS. 4B and C showing two tumors expressing notably different levels of CD44 when determined by the two techniques. Altogether these data show that the optimized protocol did affect neither cell subpopulation representation nor their antigenic properties.

### Example 15

### Quantification of Cell Surface Expression of Metabolite Transporters

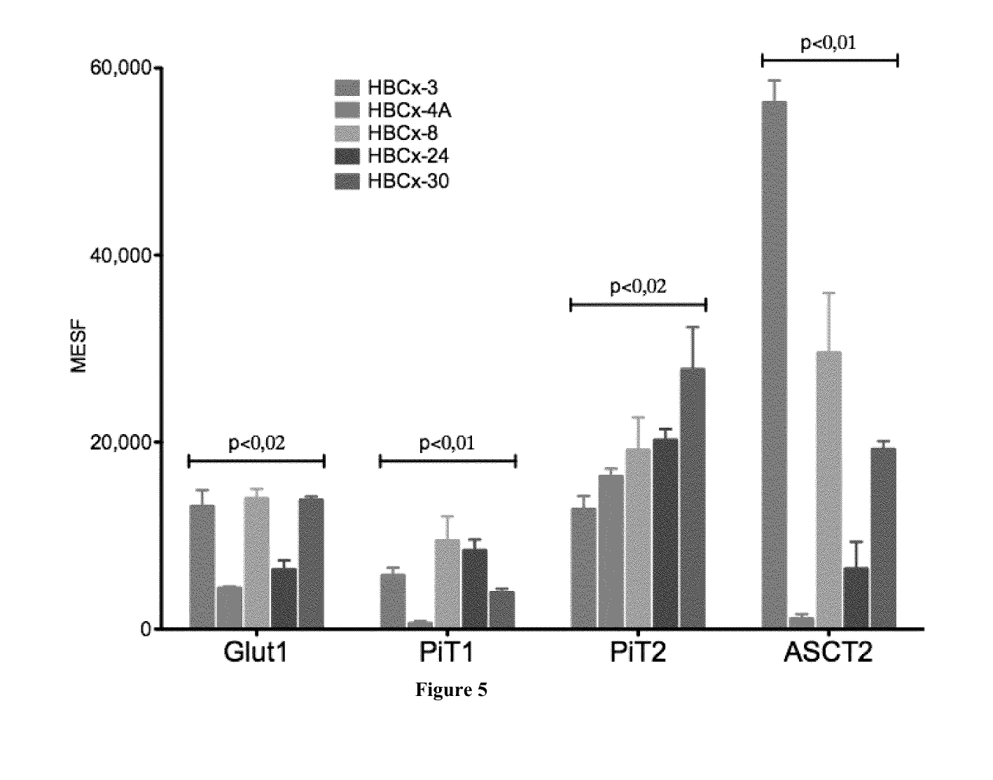

Eventually, we have monitored cell surface expression of four distinct metabolite transporters, namely Glut1, ASCT2, PiT1 and PiT2 by using RBDs derived from HTLV-2, RD114, KoERV, and AMLV envelope glycoproteins respectively. Indeed, these four transporters have been shown to be the receptors used by the aforementioned retroviruses. Their respective expressions were quantified for five human breast cancer models, i.e. HBCx-3, -4A, -8, -24 and -30, in terms of MESF (Molecular Equivalent Soluble Fluorophores (FIG. 5). Statistical analysis showed that the four transporters were highly differentially expressed in the five studied tumors. Two models out of five expressed two times less Glut1 (HBCx-4A and HBCx-24, p<0.02) compared to the three others. ASCT2 was highly variably expressed between the five models (p<0.01); notably, HBCx-3 expressed more than 20 fold much ASCT2 than HBCx-4A. PiT1 was very lightly expressed in HBCx-24 compared to the four others BC models (p<0.01). Eventually, PiT2 presented the narrower range, being expressed two fold higher in HBCx-30 compared to HBCx-3 (p<0.02). Altogether, these results highlight the fact that human breast cancers could be specifically defined by their cell surface transportome (receptor) profiling.

The clustering analysis is shown FIG. 7.

1. Recovery process of single viable cells from a solid tumor comprising two tissue dissociation steps using a non enzymatic dissociation buffer (NEDB) and an enzymatic tissue dissociation, in particular consisting of collagenase III and DNase I to obtain a mixture of isolated dead or viable cells and debris, followed by a cell purification step with a dual density Ficoll™ to eliminate red cells and debris, and thus enrich said mixture in single viable cells.

2. Recovery process of single viable cells from a solid tumor according to claim 1, comprising further an enzymatic tissue dissociation step with trypsine after said two tissue dissociation steps to obtain a mixture of isolated dead or viable cells and debris.

3. Recovery process of single viable cells from a solid tumor according to claim 1, wherein said solid tumor is a human solid tumor.

4. Recovery process of single viable cells from a solid tumor according to claim 1, wherein said solid tumor is a human solid tumor previously grafted in a mouse.

5. Recovery process of single viable cells from a solid tumor according to claim 1, wherein said human solid tumor or said grafted human solid tumor in a mouse, is a human breast cancer tumor or an UvMel melanoma.

6. Recovery process of single viable cells from a solid tumor according to claim 1, wherein said human solid tumor or said grafted human solid tumor, is a human breast cancer tumor selected from the group consisting of: HBCx-3, HBCx-4A, HBCx-8, HBCx-9, HBCx-10, HBCx-12A, HBCx-14, HBCx-22, HBCx-24, HBCx-30 and HBCx-41, or an UVmel melanoma selected from the group consisting of MP34, MP38, MP41, MP42, MP46, MP47, MP55, MP71, MP77, MP80, MM26, MM33, MM52, MP65, MM66 and MP74, in particular MM33, MP34, MP41, MP55.

7. Single viable cells obtained from a recovery process of single viable cells from a solid tumor, in particular from a recovery process of single viable cells from a solid tumor according to claim 1, in combination with at least one soluble receptor binding ligands derived from the soluble part of the glycoprotein of an enveloped virus that interacts with a cellular cognate receptor,
said receptor binding ligands containing a part or the totality of one of the receptor binding domains (RBD) of said glycoprotein, and,
said soluble receptor binding ligands being liable to interact with at least one membrane receptor of said single viable cells,
for the identification and quantification of the expression of membrane receptors present on the surface of said single viable cells, said identification and quantification taking place at a given time or during a given time interval, and allowing the clinical evaluation of patient's solid tumors relative to the diagnostic, prognostic or therapeutic response assessment.

8. Single viable cells from a solid tumor according to claim 7, wherein said receptor binding ligand is selected from the list consisting of: SEQ ID NO: 1 to 41.

9. Single viable cells from a solid tumor according to claim 7, wherein said at least one soluble receptor binding ligand is a set of two soluble receptor binding ligands, and allows the identification and the quantification of the expression of at least two membrane receptors present on the surface of single viable cells.

10. Single viable cells from a solid tumor according to claim 8, wherein said at least one soluble receptor binding ligand is a set of three to twelve soluble receptor binding ligands, in particular in particular three, four, five, six seven, eight, nine, ten, eleven, or twelve receptor binding ligands.

11. Single viable cells from a solid tumor according to claim 7, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Xenotropic Murine Leukemia Virus (NZB, Xeno, SEQ ID NO: 34), Feline Leukemia Virus C (FeLVC, SEQ ID NO: 35), Koala Retrovirus (KoRV, SEQ ID NO: 36), Porcine Endogeneous Retrovirus-A (Perv A, SEQ ID NO:37), Porcine Endogeneous Retrovirus-B (Perv B, SEQ ID NO: 38), Human T Leukemia Virus-2 (HTLV2, SEQ ID NO:40) or Bovine Leukemia Virus (BLV, SEQ ID NO: 41).

12. Single viable cells from a solid tumor according to claim 11, wherein said at least one soluble receptor binding ligand is liable to interact with at least one membrane receptor of said single viable cells and wherein said membrane receptors are selected from the list consisting in PiT2, XPR1, Pill, ASCT1, ASCT2, FLVCR1, RFT1, RFT3, Glut1.

13. Single viable cells from a solid tumor according to claim 11, wherein said tumor is a human breast cancer tumor or an UvMel melanoma.

14. Single viable cells from a solid tumor according to claim 13, wherein said tumor is a human breast cancer tumor selected from the group consisting of: HBCx-3, HBCx-4A, HBCx-8, HBCx-9, HBCx-10, HBCx-12A, HBCx-14, HBCx-22, HBCx-24, HBCx-30, HBCx-41 or an UvMel melanoma selected from the group consisting of MP34, MP38, MP41, MP42, MP46, MP47, MP55, MP71, MP77, MP80, MM26, MM33, MM52, MP65, MM66 and MP74, in particular MM33, MP34, MP41, MP55.

15. Single viable cells from a solid tumor according to claim 13, wherein said at least one receptor binding ligand is selected from the list consisting of: Amphotropic Murine Leukemia Retrovirus (AMLV, SEQ ID NO:32), Feline endogenous virus (RD114, SEQ ID NO:33), Koala Retrovirus (KoRV, SEQ ID NO: 36) or Human T Leukemia Virus-2 (HTLV2, SEQ ID NO:40).

16. Single viable cells from a solid tumor according to claim 13, wherein said tumor is a human breast cancer tumor selected from the group consisting of: HBCx-3, HBCx-4A, HBCx-8, HBCx-24, HBCx-30.

17. Single viable cells from a solid tumor according to claim 13, wherein said at least one soluble receptor binding ligand is liable to interact with at least one membrane receptor of said single viable cells, wherein said membrane receptor is ASCT2, and said tumor is a HBCx-3 human breast cancer tumor, ASCT2 receptor being overexpressed

18. Single viable cells from a solid tumor according to claim 13, wherein said at least one soluble receptor binding ligand is liable to interact with at least one membrane receptor of said single viable cells, wherein said membrane receptor is Glut1 and PiT1 and ASCT2, said tumor is a HBCx-4A or HBCx-24 human breast cancer tumor, Glut1, Pill and ASCT2 receptors being underexpressed.

19. Single viable cells from a solid tumor according to claim 13, wherein said at least one soluble receptor binding ligand is liable to interact with at least one membrane receptor of said single viable cells, wherein said membrane receptor is PiT2, and said tumor is a HBCx-30 human breast cancer tumor, PiT2 receptor being overexpressed.

20. Process of diagnostic or prognostic of solid cancer tumors in a patient comprising:
a. Sampling of a biological material suspected to be a solid cancer tumor from a patient,
b. Optionally, grafting said tumor in a mouse,
c. Implementation of a recovery process of single viable cells according to claim 1, from a human solid tumor of step a, or a human solid tumor from mouse of step b after excision from the mouse,
d. Contacting said single viable cells from a human solid tumor from a patient or a human solid tumor from mouse of step c, with at least one soluble receptor binding ligands derived from the soluble part of the glycoprotein of an enveloped virus that interacts with a cellular cognate receptor,
e. identifying and quantifying the expression of membrane receptors present on the surface of said single viable cells of step d,
f. comparing the expression of membrane receptors obtained in each said single viable cells of step e with a respective control,
g. an overexpression and/or a underexpression of membrane receptors in each said single viable cells compared to their respective control being indicative of a solid cancer tumor.

21. Process of a therapeutic response assessment in a patient having a treatment against solid cancer tumors comprising:
a. Sampling of a tumor to be analyzed from a patient suffering from a solid cancer tumor and treated with an anticancer drug,
b. Optionally, grafting said tumor in a mouse,
c. Implementation of a recovery process of single viable cells according to claim 1, from a human solid tumor of step a, or a human solid tumor from mouse of step b after excision from the mouse,
d. Contacting said single viable cells from a human solid tumor from a patient or a human solid tumor from mouse of step c, with at least one soluble receptor binding ligands derived from the soluble part of the glycoprotein of an enveloped virus that interacts with a cellular cognate receptor,
e. identifying and quantifying the expression of membrane receptors present on the surface of said single viable cells of step d,
f. comparing the expression of membrane receptors obtained in step e with the one obtained before treatment of said patient or said mouse,
g. an increase of the expression of an underexpressed membrane receptor or a decrease of an overexpressed membrane receptor obtained in each said single viable cells of step e compared respectively to the one obtained before treatment being respectively indicative of a therapeutic response by the patient or the mouse to an anticancer treatment.

22. Screening process of a drug liable to treat a solid cancer tumor comprising:
a. Sampling of a tumor to be analyzed from a patient suffering from a solid cancer tumor,
b. Optionally, grafting said tumor in a mouse, and treating the mouse with a drug to test,
c. Implementation of a recovery process of single viable cells according to claim 1, from a human solid tumor of step a, or a human solid tumor from a mouse of step b after excision from the mouse,
d. Culturing said single viable cells from a human solid tumor of step c and treating them with a drug to test,
e. Contacting said single viable cells from a human solid tumor from a patient of step d or a human solid tumor from mouse of step c, with at least one soluble receptor binding ligands derived from the soluble part of the glycoprotein of an enveloped virus that interacts with a cellular cognate receptor,
f. identifying and quantifying the expression of membrane receptors present on the surface of said single viable cells of step e,
g. comparing the expression of membrane receptors obtained in step f with the one obtained before treatment of said single viable cells from a human solid tumor or from human solid tumor from a mouse,
h. an increase of the expression of an underexpressed membrane receptor or a decrease of an overexpressed membrane receptor obtained in each said single viable cells of step f compared respectively to the one obtained before treatment being respectively indicative of a drug liable to treat a solid cancer tumor.
